(19) United States
(12) Patent Application Publication
(10) Pub. No.: US 2026/0327336 A1
(43) Pub. Date: Oct. 22, 2026

(54) ULTRASTABLE ANTIBODY FRAGMENTS WITH A NOVEL DISULDIDE BRIDGE

(71) Applicant: Turun yliopisto, Turun yliopisto (FI)

(72) Inventors: Urpo LAMMINMÄKI, Turun yliopisto (FI); Eeva-Christine BROCKMANN, Turun yliopisto (FI); Anna HAAPALINNA, Turun yliopisto (FI)

(21) Appl. No.: 19/117,302

(22) PCT Filed: Oct. 3, 2023
(86) PCT No.: PCT/FI2023/050564

§ 371 Date: Mar. 31, 2025

(30) Foreign Application Priority Data
Oct. 3, 2022 (FI) 20225887

(51) Int. Cl. C07K 16/32; C40B 40/10; C40B 50/06; G01N 33/543; G01N 33/68

(52) U.S. Cl. CPC C07K 16/32; G01N 33/54313; G01N 33/68; C07K 2317/24; C07K 2317/565; C07K 2317/622; C07K 2317/92; C07K 2317/94; C40B 40/10; C40B 50/06; G01N 2333/71

(57) ABSTRACT

The present invention relates to a recombinant library of particles, the library displaying a plurality of single-chain variable fragments (scFvs) against various antigens, such as HER2. The displayed single-chain variable fragments are characterized by improved stability owing to a non-native interdomain disulfide bridge in a novel position. The invention also relates to a method of constructing such a library, uses of the library to obtain stabilized scFvs with desired antigenbiding properties, as well as to such scFvs.

### FIELD OF THE INVENTION

The present invention relates to recombinant antibody fragments, more specifically to single-chain variable fragments with improved stability owing to a non-native interdomain disulfide bridge in a novel position. The invention also relates to recombinant expression libraries of such antibody fragments and to methods of constructing such libraries and uses thereof to select stable antibody fragments with desired antigen-binding properties.

### BACKGROUND OF THE INVENTION

Single-chain fragment variables (scFvs) have multiple advantages over full-length antibodies. They can be cost-efficiently expressed in micro-organisms with high yields. They are small, which allows them to reach cryptic (hidden) epitopes. In addition, due to their small size, they display improved pharmacokinetic properties—scFv fragments penetrate more rapidly and evenly to tissues (such as tumors) and have more rapid clearance, which may be beneficial in radiotherapy and diagnostic in vivo applications. The small size of scFvs enables them to be screened and selected by in vitro display methods such as phage display that avoid animal immunizations. Moreover, scFvs allow production of various types of advanced antibody formats by genetically conjugating them to other entities, such as antibody fragments or intact antibodies.

Unfortunately, conventional scFv fragments are prone to variable and dynamic oligomerization, and/or domain swapping phenomenon, in which two or more scFvs are paired intermolecularly. Oligomerization has many consequences on the functionality and the stability of scFv. For example, it can increase the aggregation propensity of the scFvs, and thus bring various handling and storage problems. It also complicates the medical use of scFvs, as well as their use in vitro and diagnostic assay development as oligomeric scFv molecules have altered affinity and activity against antigen due to an avidity effect. Oligomers may also cross-link targets in vivo, causing altered pharmacodynamic effects.

High stability is one of the most significant demands for antibodies and antibody fragments in biomedical applications. Unfortunately, many scFvs are less stable than the corresponding full-length antibodies: many scFvs are prone to denaturation and aggregation caused by exposure of the hydrophobic residues at the V_{H}-V_{L} interface. Generally, scFvs possess also lower thermostability than the corresponding intact antibodies or Fab fragments.

Stabilization of scFvs can be achieved, at least to some extent, by engineering stabilizing disulfide bridges into the scFv molecules. Indeed, various positions in V_{H} and V_{L} have previously been tested for the introduction of a disulfide bridge for stabilization of scFvs. However, the stability varies by this approach and therefore there is still need for means to provide scFvs with improved stability.

### SUMMARY

The present invention provides a disulfide stabilized single-chain fragment variable (ds-scFv), which comprises a heavy chain variable domain (V_{H}) comprising complementary determining regions CDR-H1, CDR-H2 and CDR-H3, and a light chain variable domain (V_{L}) comprising complementary determining regions CDR-L1, CDR-L2 and CDR-L3, wherein the V_{H} and the V_{L} are joined by a peptide linker in either orientation, and wherein the CDR-L1 and the CDR-H3 are attached to each other through an interdomain disulfide bridge and wherein either the V_{H} or the V_{L} has been engineered to lack the native conserved intradomain disulfide bridge. In this context, the native intradomain disulfide bridge refers in particular to a bridge formed between cysteine residues L23 and L88 in V_{L} and/or to a bridge between cysteine residues H22 and H92 in V_{H} according to the Kabat numbering scheme. Preferably, the native intradomain disulfide bridge is lacking from the variable domain placed after the peptide linker.

In some embodiments, the interdomain disulfide bridge is artificially introduced.

In some further embodiments, the artificially introduced disulfide bridge is formed between a cysteine residue in the CDR-H3 at position −4 counted from conserved tryptophan H103 in the FR-H4 and a cysteine residue in the CDR-L1 at position L34 according to the Kabat numbering scheme.

In some embodiments, the peptide linker is at least 12 amino acids long.

In some embodiments, the CDR sequences of the V_{H} and/or the V_{L} are obtained from a natural diversity except for cysteines at the residue −4 counted from conserved tryptophan H103 of the FR-H4 and at the residue L34 of the CDR-L1 according to the Kabat numbering scheme. In some other embodiments, the CDR sequences of the V_{H} and/or the V_{L} are designed completely or partly in silico, whereas in some further embodiments, one or more of the CDR sequences of the V_{H} and/or the V_{L} contain one or more randomized amino acids. In both cases, a cysteine residue in the CDR-H3 at position −4 counted from conserved tryptophan H103 in the FR-H4 and a cysteine residue in the CDR-L1 at position L34 according to the Kabat numbering scheme must be present.

In some embodiments, the ds-scFv comprises a humanized framework or a framework derived from a human antibody. In some more specific embodiments, the V_{H} domain of the ds-scFv comprises a framework comprising FR-H1 of SEQ ID NO:1, FR-H2 of SEQ ID NO:2, FR-H3 of SEQ ID NO:3 and FR-H4 of SEQ ID NO:4 in this order, and the V_{L} domain comprises a framework comprising FR-L1 of SEQ ID NO:5, FR-L2 of SEQ ID NO:6, FR-L3 of SEQ ID NO:7 and FR-L4 of SEQ ID NO:8 in this order. In some further embodiments, the framework is a functionally equivalent conservative sequence variant of the sequences mentioned. In accordance with some aspects of the invention, namely ds-scFvs lacking the other native intradomain disulfide bridge, the framework has been engineered such that it does not contain both of the native cysteines at positions corresponding to residue 22 of SEQ ID NO:1 and residue 30 of SEQ ID NO:3 or both of the native cysteines at positions corresponding to residue 23 of SEQ ID NO:5 and residue 32 of SEQ ID NO:7.

In some embodiments, the V_{H} domain of the ds-scFv comprises an amino acid sequence set forth in SEQ ID NO:48, while the V_{L} domain comprises an amino acid sequence set forth in SEQ ID NO:49, or a functionally equivalent conservative sequence variant of the sequences mentioned. In accordance with some aspects of the invention, namely ds-scFvs lacking the other native intradomain disulfide bridge, said SEQ ID NO:48 has been engineered not to contain both of the native cysteines at positions corresponding to residues 22 and 98 thereby preventing the formation of the native intradomain disulfide bridge within V_{H}, or said SEQ ID NO:49 has been engineered not to contain both of the native cysteines at positions corresponding to residues 23 and 88 thereby preventing the formation of the native intradomain disulfide bridge within V_{L}.

In some further embodiments of the above, the ds-scFv is an anti-HER2 ds-scFv. Preferably, anti-HER2 ds-scFv comprises a CDR-H1 having an amino acid sequence of SEQ ID NO:12, a CDR-H2 having an amino acid sequence of SEQ ID NO:13, a CDR-H3 having an amino acid sequence of SEQ ID NO:14, a CDR-L1 having an amino acid sequence of SEQ ID NO:15, a CDR-L2 having an amino acid sequence of SEQ ID NO:16 and a CDR-L3 having an amino acid sequence of SEQ ID NO:17. In some more specific embodiments, the anti-HER2 ds-scFv comprises an amino acid sequence of SEQ ID NO: 20 or of SEQ ID NO: 21.

The present invention also provides a molecular entity comprising one or more ds-scFv units according to various embodiments of the invention, as well as use of the ds-scFv according to any embodiments of the invention for constructing such a molecular entity. In some embodiments, the molecular entity is a bispecific antibody, a diabody, a multispecific antibody, a CAR-T cell, a bispecific T-cell engager (BiTE), or a construct comprising the ds-scFv of the invention fused to a moiety such as a fragment crystallizable (Fc) part of an antibody or an alternative protein scaffold-based affinity reagent, such as Design Ankyrin Protein (DARPin), nanobody or an affibody. The molecular entity may also be, for example, a pharmaceutically active agent, a drug, a radioisotope, an enzyme or a chelating agent.

In still further aspect, the present invention provides a nucleic acid molecule encoding the ds-scFv according to any embodiment of the invention.

In even further aspects, the invention provides a particle that displays on its surface the ds-scFv according to any embodiments of the invention, as well as a library of such particles, the library displaying a plurality of different ds-scFvs of the invention. Preferably, the particle is a phage particle, a yeast cell, a bacterial cell, a mammalian cell or a ribosome.

Moreover, the present invention provides a method of making a library of particles, wherein the library displays a plurality of different ds-scFvs according to various embodiments of the invention. The method comprises engineering a plurality of first nucleic acids that encode different heavy chain variable domain (V_{H}) polypeptides all having a cysteine residue at position −4 counted from conserved tryptophan H103, according to the Kabat numbering scheme, and engineering just one or a plurality of second nucleic acids that encode different light chain variable domain (VL) polypeptides all having a cysteine residue at position L34 according to the Kabat numbering scheme. Each one of the plurality of first nucleic acids is then cloned with the one and only second nucleic acid or with one of the plurality of second nucleic acids into an expression vector in either order to contain nucleic acids that encode an intervening peptide linker, including but not limited to peptide linkers such as those comprising of consisting SEQ ID NOs: 9-11. If a plurality of second nucleic acids is used, the combinations of the first and the second nucleic acids are random. The cloning results in a plurality of different vectors which are then expressed on particles, thereby generating a first library of particles, each particle displaying a different ds-scFv polypeptide comprising a V_{H} and a V_{L} segment encoded by the first and the second nucleic acids. Preferably, the first library of particles is a phage display library.

In the method, either the first or the second nucleic acids are engineered to lack the native intradomain disulfide bridge in the encoded polypeptide. In this context, the native intradomain disulfide bridge refers in particular to a bridge formed between cysteine residues L23 and L88 in V_{L} and/or to a bridge between cysteine residues H22 and H92 in V_{H} according to the Kabat numbering scheme. Preferably, the native intradomain disulfide bridge is lacking from the variable domain placed after the peptide linker in the encoded polypeptide.

In some embodiments, either the first or the second nucleic acids, or both, may be engineered such that the encoded polypeptide has one or more CDR loops with one or more randomized amino acids, provided that the cysteines participating in the formation of the interdomain disulfide bridge and in the remaining intradomain disulfide bridge remain unaltered.

In some embodiments, the method may comprise introducing further diversity into a subset of the first library of particles having desired target-binding properties, thereby creating a second library of particles, preferably a library of cell particles, more preferably a library of yeast or mammalian cells, even more preferably a library of mammalian cells. Said further diversity may be introduced, for example, by mutagenesis or domain shuffling of either the V_{H} or V_{L}. Preferably, shuffling of the variable domain located after the peptide linker is employed. Notably, the mutagenesis or the domain shuffling preferably results in reintroduction of the cysteines that form the native intradomain disulfide bridges in the polypeptides displayed by the second library.

In some embodiments, all or part of the plurality of the first nucleic acids, and/or the one second nucleic acid, or all or part of the plurality of the second nucleic acids, as the case may be, are designed artificially and/or obtained synthetically. In some other embodiments, all or part of the plurality of the first nucleic acids, and/or the one second nucleic acid, or all or part of the plurality of the second nucleic acids, as the case may be, are obtained from a natural diversity except for the engineered nucleotides corresponding to cysteines in the CDR-H3 at the residue −4 counted from tryptophan H103 in the FR-H4 and at the residue L34 of the CDR-L1 in the encoded polypeptide.

Further aspects, embodiments and details are set forth in the following figures, detailed description, and examples.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 is a schematic presentation of the scFv constructs used in the examples.

FIG. 2 shows an SDS-PAGE of the purified scFv variants LH_SS−, LH_SSC, HL_SSC, HL_S-C, and LH_S-C expressed in E. coli. The variants with an interdomain disulfide bridge migrate faster in nonreduced form (N). Ready Blue Protein Gel Stain was used for staining the gels. Imaging was performed with ChemiDoc, ImageLab 5.2.1 software (Bio-Rad). Precision Plus Protein Dual Color Standards (Bio-Rad) was used as a marker (lane 1). R=samples reduced with 5% β-mercaptoethanol. N=samples run in nonreduced form.

FIG. 3 shows SDS-PAGEs of the purified scFv variants LH_SS−, HL_SS−, LH_SSC, HL_SSC, HL_S-C, LH_S-C, LH_SS+, and HL_SS− Expressed in ExpiCHO. The variants with an interdomain disulfide bridge migrate faster in nonreduced form (N). Ready Blue Protein Gel Stain was used for staining the gels. Imaging was performed with ChemiDoc, ImageLab 5.2.1 software (Bio-Rad). Precision Plus Protein Dual Color Standards (BioRad) was used as a marker (lane 1). R=samples reduced with 5% β-mercaptoethanol. N=samples run in nonreduced form.

FIG. 4 demonstrates immunoreactivity of the scFv constructs against HER2. Each bar represents the mean of 3 values±standard deviation. The specific signal was calculated by reducing the signal obtained from streptavidin wells from the signal obtained from wells containing biotinylated HER2. Concentrations of the scFvs: 10 nM (black), 100 nM (patterned), and 300 nM (white).

FIG. 5 shows binding of scFv constructs produced in E. coli to HER2 detected by bio-layer interferometry (BLI) (OctetRED384). The y-axis represents the binding (nm) and the x-axis time (s).

FIG. 6 shows binding of scFv constructs produced by Expi-CHO™ system to HER2 detected by bio-layer interferometry (BLI) (OctetRED384). The y-axis represents the binding (nm) and the x-axis time (s).

FIGS. 7A and 7B show derivative plots showing the midpoint of the melt transition (Tₘ) of the scFvs expressed in LH (7A) and HL (7B) orientation. LH_SS− and HL_SS− (black circles), LH_S-C and HL_S-C (white triangle), LH_SSC and HL_SSC (black square), LH_SS+ and HL_SS+ (white rhombus). Negative control (buffer only) is marked as a straight line in the figure.

FIG. 8 illustrates influence of the signal sequence and scFv construct on phage display levels as assessed by binding to protein L. The specific signals informed as time-resolved fluorescence counts represent the mean of three values±standard deviation. Black: phagemid vector pEB32x, White: phagemid vector pEB3V3 with modified pelB signal sequence.

FIG. 9 illustrates influence of the effect of signal sequence and scFv construct on the binding to HER2. All the phage stocks were used with 5×10⁹ cfu/ml phages in the test. Each bar represents the mean of three values±standard deviation. Black: pEB32x, White: pEB3V3 with modified pelB signal sequence.

FIG. 10 is a schematic presentation of the scFv library construction. The CDR-H3 was randomized with the NNS codon. Two PCR products, A and B, were produced with the forward primer WO375 and reverse primer HL_S-C Rev, and the forward primers HL_S-C 13-19 aa loop and reverse primer HS076 new seq rev, respectively. The PCR A and B products were digested by LguI to form cohesive ends and ligated to create an scFv library by modified FASTR reactions.

FIG. 11 shows binding of the loop library phage stocks to protein L in phage immunoassay illustrating the phage display levels of scFv. Each bar represents a mean of three values±standard deviation.

FIG. 12 shows phage immunoreactivity assay results of scFv libraries panned against biotinylated HER2 as specific signals to HER2. Each bar represents the mean of three values±standard deviation. Black: panning round 1; patterned: panning round 2; white: panning round 3.

FIG. 13 demonstrates that individual clones isolated from the libraries after the panning round 3 produce specific signals to HER2. Each square represents a single clone/well in the 96-well plate. The shown values were calculated by reducing the signal obtained from streptavidin wells from the signal obtained from wells containing biotinylated HER2.

FIG. 14 shows single clone sequence alignment of CDR-H3 loop regions of the HER2 specific clones isolated after the panning round 3. Randomized region is in bold. The nonnative cysteine (at position H100B in the CDR-H3 of the template ds-ScFv HL_S-C) is indicated by *.

### DEFINITIONS

Before the invention is described, it is to be understood that this disclosure is not strictly limited to any particular compositions, reagents, devices, protocols or methodology described herein, as such may vary. It is also to be understood that the terminology used herein is for the purpose of describing particular embodiments only, and is not intended to be limiting, since the scope of the present invention will be limited only by the appended claims.

It is also to be noted that, unless defined otherwise, all technical and scientific terms used herein have the same meaning as commonly understood by one of ordinary skill in the art to which this invention belongs.

It is further to be noted that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination. Moreover, any features, details or embodiments disclosed in the context of ds-scFvs provided herein apply also to a library of such ds-scFvs provided herein, and vice versa, if applicable and even if not repeated.

As used herein and in the appended claims, the singular forms “a”, “an” and “the” mean one or more. Thus, a singular noun, unless otherwise specified, carries also the meaning of the corresponding plural noun, and vice versa. As such, the terms “a”, “an”, “one or more” and “at least one” can be used interchangeably.

The term “and/or” in a phase such as “X and/or Y” shall be understood to mean either “X and Y” or “X or Y” and shall be taken to provide explicit support for both meanings or for either meaning.

The terms “comprising”, “including” and “having” can be used interchangeably.

As used herein, the term “antibody” refers to the structure of immunoglobulin G comprising two identical heavy chains (~50-60 kDa) and two identical light chains (~23 kDa) made up of globular structural motifs called Ig-fold domains. In the heavy chain, there are three constant Ig-fold domains (C_{H1}, C_{H2}, and C_{H3}) and one variable Ig-fold domain (V_{H}), whereas the light chains consist of one constant (C_{L}) and one variable (V_{L}) Ig-fold domain. Each domain consists of two anti-parallel β-sheets that are formed of seven-to-nine anti-parallel β-strands connected by loops. The Ig-fold domain is stabilized by a highly conserved intradomain disulfide bridge formed between cysteine residues in the two anti-parallel β-sheets. Typically, two or four covalent disulfide bridges in the hinge region located between C_{H1} and C_{H2} connects the two heavy chains, while the heavy and light chains are connected through a covalent disulfide bridge linking the C_{H1} and C_{L}. As used herein, the terms “V_{H}” and “V_{L}” are interchangeable with the terms “V_{H} domain” and “V_{L} domain”, respectively.

As used herein, the term “complementary-determining region” (CDR) refers to highly variable regions in the variable domains of an antibody. There are three CDRs in each variable domain: CDR-L1, CDR-L2, and CRD-L3 in the V_{L} domain, and CDR-H1, CDR-H2, and CDR-H3 in the V_{H} domain. All the CDRs are collectively involved in antigen recognition and binding. However, the CDR-H3 is generally considered as the most important CDR engaged in antigen-binding as it is the most variable in loop length and sequence. As used herein, the terms “CDR” and “CDR loop” are interchangeable.

As used herein, the term “framework” (FR) refers to a non-CDR portion of a variable domain (RB-sheets and non-hypervariable loops). It provides structural support to the antigen-binding site but also affects the CDR loop conformations. In the primary structure of an antibody (i.e., its linear amino acid sequence), the term “framework” refers to amino acid sequences interposed between the CDRs. Accordingly, there are four framework regions in each variable domain: FR-L1, FR-L2, FR-L3 and FR-L4 in the V_{L} domain, and FR-H1, FR-H2, FR-H3 and FR-H4 in the V_{H} domain.

As used herein, the term “humanized framework” refers to a framework of human origin which typically is engineered to contain some amino acid alterations as compared to the original framework of human origin. Since the amino acid alterations are non-native to the original framework of human origin, they can be denoted as amino acids of non-human origin. Typically, humanized frameworks are used in humanized antibodies, i.e. in antibodies wherein CDR regions of non-human origin are integrated into a framework of human origin. The purpose of humanizing the framework, i.e. introducing amino acids of non-human origin, is to assure that functional properties of the humanized antibody correspond to those of the parental non-human antibody from which the CDR sequences have been derived. Those skilled in the art know how to humanize a given framework for a given parental non-human antibody.

Human immunoglobulin V_{L} or V_{H} domains exist in different subtypes as is generally known in the art. In one embodiment, for V_{L}, the framework corresponds essentially to the framework of subtype kappa I as is known to those skilled in the art. In one embodiment, for V_{H}, the framework corresponds essentially to the framework of subtype III as is also known to those skilled in the art. In some further embodiments, the framework can be a “consensus framework”, i.e., a framework that represents the most frequently occurring amino acid residues in the human immunoglobulin V_{L} or V_{H} framework sequences within each existing V_{L} and V_{H} subtypes.

As used herein, the term “conservative sequence variant” refers to an amino acid sequence comprising modifications which do not significantly alter structural or functional properties of the antibody in question. Conservative amino acid sequence variants include variants arising from amino acid substitutions with similar amino adds. As is well known in the art, said similarity may be determined on the basis of similarity in polarity, charge, solubility, hydrophobicity, hydrophilicity, and/or the amphipathic nature of the residues involved. Conservative amino acid sequence variants also include variants comprising small amino acid deletions and/or insertions. Preferably, the conservative sequence variants encompassed by the invention can be denoted as “functionally equivalent conservative sequence variants”. Those skilled in the art can easily determine whether or not given sequence variants are functionally equivalent or not.

All numbering of amino acid positions within variable domains as used herein, as well as identification of CDRs, is in accordance with the Kabat numbering scheme unless otherwise specified. The Kabat numbering scheme is well known to those skilled in the art. Accordingly, the expression “H100B-L34”, for example, refers to a polypeptide bearing a cysteine in CDR-H3 at position H100B and a cysteine in CDR-L1 at position L34 according to the Kabat numbering scheme. Likewise, the expression “H44-L100” refers to a polypeptide bearing a cysteine in FR-H2 at position H44 and a cysteine in FR-L4 at position L100 according to the Kabat numbering scheme. It is to be noted that the assignment of positions according to the Kabat numbering scheme refers to certain amino acid positions defined by sequence conservativeness and not to actual amino acid positions in the linear amino acid sequence of a given antibody.

In more general terms, H100B corresponds to an amino acid position −4 counted from highly conserved tryptophan H103 in a V_{H} polypeptide, which tryptophan is the first amino acid of FR-H4. Such an indirect definition for the CDR-H3 cysteine participating in the non-native interdomain disulfide bridge is need since, as set forth later in this description, the length of the CDR-H3 loop may vary, hence changing the Kabat numbering of the cysteine residue. Notably, the expression “position −4 counted from tryptophan H103 in the FR-H4” and the like, are not affected by sequence variations preceding said position −4.

It is to be noted that in the context of the present invention, the last two amino acids of FR-H3 according to the Kabat numbering scheme are considered as the two first amino acid residues of CDR-H3. Therefore, the FR-H3 is two amino acid residues shorter, whereas the CDR-H3 loop is two amino acid residues longer than conventionally considered by the Kabat numbering scheme. In other words, using the Kabat numbering, the FR-H3 is herein defined by amino acid residues H66-H92, while the CDR-H3 is defined by amino acid resides H93-H102. Corresponding conventional definitions are H66-H94 and H95-H102, respectively. It is to be noted that the Kabat numbering scheme permits length variation within the CDR-H3 without affecting the definition of CDR-H3 by Kabat numbers. In other words, despite any variation in the loop length, CDR-H3 is composed of amino acids H93-H102 according to the Kabat numbering scheme.

As used herein, the term “fragment variable” (Fv) refers to an antibody fragment composed of V_{L} and V_{H} domains. The Fv fragment is intrinsically unstable as the V_{L} and V_{H} domains are not covalently bound to each other through a peptide linker or a disulfide bond.

As used herein, the term “single-chain fragment variable” (scFv) refers to a recombinant Fv fragment composed of the V_{L} and V_{H} domains connected by a flexible peptide linker, which stabilizes the structure. One of the most significant drawbacks of conventional scFvs hindering their full potential is that, despite having a stabilizing peptide linker, their stability may still be an issue. Moreover, many conventional scFvs are prone to aggregation and to variable and dynamic oligomerization. In addition, they possess lower thermostability than the corresponding full antibodies.

As used herein, the term “disulfide stabilized Fv fragment” (dsFv) refers to an antibody fragment composed of V_{L} and V_{H} domains that are connected to each other through a disulfide bridge.

As used herein, the term “disulfide-stabilized single-chain fragment variable” (ds-scFv) refers to a recombinant Fv fragment composed of the V_{L} and V_{H} domains connected by a flexible peptide linker in either orientation (V_{L}-linker-V_{H} or V_{H}-linker-V_{L}) and further stabilized by an artificial interdomain disulfide bridge. In the present invention, the stabilizing interdomain disulfide bridge is in a novel position between CDR-H3 and CDR-L1 loops. Owing to its position, the interdomain disulfide bridge of the invention may also be denoted as an H3/L1-interloop disulfide bridge.

As used herein, the term “disulfide bridge” refers to a covalent bond between two sulfur atoms (—SS—) formed by the coupling of two thiol (—SH) groups. Cysteine, one of 20 proteinogenic amino acids, has a —SH group in its side chain, and can easily be dimerized to cystine in aqueous solution by forming a disulfide bond.

Two types of disulfide bridges in terms of their position are relevant for the present invention. The first type, namely “intradomain disulfide bridge” refers to a highly conserved native disulfide bridge formed between cysteine residues in two anti-parallel β-sheets within V_{H} and V_{L}. Both in V_{H} and V_{L}, the intradomain disulfide bridge joins the first and the third framework regions, i.e., FR-H1 and FR-H3 in V_{H} and FR-L1 and FR-L3 in V_{L}. In V_{H}, the cysteines forming the bridge are at positions H22 and H92, whereas in V_{L} the cysteines are at positions L23 and L88 according to the Kabat numbering scheme.

The second type of disulfide bridges, namely “interdomain disulfide bridge” refers to a non-native disulfide bridge between V_{H} and V_{L}, more specifically between CDR-H3 and CDR-L1 loops. The disulfide bridge is “artificial” in the sense that the cysteine residues forming the bridge have been artificially introduced (i.e., engineered) by recombinant techniques into the scFv in question thereby giving rise to a corresponding ds-scFv. It is to be understood that there is nothing artificial or non-natural in the cysteine residues per se or in the disulfide bridge formed between the cysteine residues. As used herein, the term “artificial” is interchangeable with the term “non-native”. In the context of ds-scFvs of the invention comprising a stabilizing disulfide bridge between CDR-L1 and CDR-H3 loops, the term “interdomain disulfide bridge” is interchangeable with the term “interloop disulfide bridge”.

In accordance with what is stated above, ds-scFvs of the invention contain three disulfide bridges, one within V_{H}, one within V_{L} and one between V_{H} and V_{L}. However, some embodiments of the invention relate to so-called intermediate ds-scFvs comprising one interdomain and one intradomain disulfide bridge only, as is explained in more detail later in this description.

As used herein, the term “transition temperature” (Tₘ) refers to a temperature at which 50% of a macromolecule, e.g., an antibody, becomes denatured, and is considered to be the standard parameter for describing the thermal stability of a protein.

As used herein, the term “Trastuzumab” (also known as hu4D5) refers to a recombinant humanized version of the murine anti-HER2 antibody 4D5. Trastuzumab has been approved for the treatment of HER2 receptor-positive breast and stomach cancers. An scFv fragment of Trastuzumab (hu4D5-8) also exists.

As used herein, the term “human epidermal growth factor receptor 2” (HER2, also known as neu) refers to a member of the human epidermal growth factor receptor (HER/EGFR/ERBB) family. It is an oncogene found on the surface of all breast cells. Amplification or over-expression of HER2 has been shown to play an important role in the development and progression of certain aggressive types of breast cancer. It has become an important biomarker and target of therapy for approximately 30% of breast cancer patients. Over-expression of HER2 is also known to occur in ovarian cancer, stomach cancer, adenocarcinoma of the lung and aggressive forms of uterine cancer.

As used herein, the term “recombinant expression library” refers to a collection of antibodies or antibody fragments, such as ds-scFvs of the invention comprising one interdomain and two intradomain disulfide bridges or an intermediate ds-scFv comprising one interdomain and one intradomain disulfide bridge, displayed on heterologous host particles (such as on phage particles, on ribosomes or on a cell surface, e.g., on yeast cells, bacterial cells or mammalian cells), or expressed in vitro. The number of different antibodies or antibody fragments in such a library is typically >1E4, more preferably >1E5, even more preferably >1E6, even more preferably >1E7, even more preferably >1E8, even more preferably >1E9, and most preferably >1E10. As known in the art, the diversity may depend on the display system in question. For example, it may be difficult to obtain diversity higher than 1E5 in mammalian cell expression libraries.

As used herein, the “E” in an expression such as “1E5” refers to exponent and indicates that the number should be multiplied by 10 raised to the power of the following number. In other words, the expression “1E5”, for example, is equivalent to the expression “1×10⁵” and equals 100,000.

### DETAILED DESCRIPTION

The present invention relates to recombinant antibody fragments, more specifically single-chain variable fragments with improved stability owing to an interdomain disulfide bridge in a novel position, namely between CDR-H3 and CDR-L1. Preferably, the interdomain disulfide bridge is formed through cysteines at position −4 counted from tryptophan H103 and at position L34 according to the Kabat numbering scheme.

In addition to the novel stabilizing interdomain disulfide bridge, ds-scFvs of the invention may contain two native intradomain disulfide bridges, one within V_{H} and one within V_{L}. However, in some aspects of the invention, the present ds-scFvs have been engineered to contain only either one of said native intradomain disulfide bridges.

Engineering either V_{H} or V_{L} such that it lacks its native intradomain disulfide bridge can be achieved by mutating either one or preferably both of the cysteine residues that would normally participate in the disulfide bridge formation, using techniques readily available in the art. To be more specific, one or both of said cysteine resides are substituted with another (i.e. non-cysteine) amino acid. In some embodiments, said non-cysteine amino acid is selected from Phe, Met, Tyr and Gly, more preferably from Leu and Ile, even more preferably from Ala and Val. This applies to all instances referred to hereinbelow which concern present ds-scFvs engineered to contain only one native intradomain disulfide bridge, including specifically mentioned SEQ ID NOs.

The CDR loops of the present ds-scFvs may vary both in terms of amino acid composition and length. Diversity of the CDRs may be derived from natural or non-natural sources, or both. Natural sources include B-cells of immunized or nonimmunized human or animal subjects, whereas non-natural diversity may be designed in silico and the genetic material be synthetized. The natural and in silico designed CDR diversity may also be combined with or without CDR randomization. Moreover, in some embodiments, the CDR sequences correspond to those of existing antibodies or antigen-binding fragments thereof, such as scFvs or Fabs. Notably, the source of one or more heavy chain CDRs can differ from the source of one or more light chain CDRs.

In some embodiments, the ds-scFvs of the invention comprise a CDR-H3 region whose length is at least 12 amino acids, and in some specific embodiments 13 amino acids. Such a CDR-H3 region is particularly suitable for incorporation of the stabilizing interdomain disulfide bridge of the invention between CDR-L1 and CDR-H3.

For CDR randomization, one or more CDR loops are usually randomized at positions most likely to contribute to antigen recognition and binding. Therefore, the order of preference for said randomization is usually CDR-H3, either one or both of CDR-H1 and CDR-H2, either one or both of CDR-L1 and CDR-L3, and CDR-L2. It is to be noted that the order of preference is not intended to be limiting, and the CDR loops may be selected for randomization independently from each other. The number of randomized amino acid positions is not limited. It is to be understood that the randomization is not limited to amino acid substitutions at given amino acid positions but may also involve amino acid insertions and/or deletions at said positions thereby resulting in potential changes in the CDR loop lengths.

For CDR-H3, the number of randomized amino acid positions is preferably at least three, more preferably at least four, even more preferably at least six, depending on the extent of diversification desired. Preferably, the amino acid alterations are at positions upstream from the cysteine at the residue −4 counted from conserved tryptophan H103 of the FR-H4 according to the Kabat numbering scheme. It is important that the CDR randomization does not involve said cysteine because otherwise formation of the interdomain disulfide bridge would not be enabled. For CDR-H1, the number of randomized amino acid positions is typically at least one, preferably two or more, if any. For CDR-H2, the number of randomized amino acid positions is typically at least two, preferably 4 or more, more preferably six or more, if any.

For CDR-L1, the number of randomized amino acid positions is preferably at least one, if any. It is important that the CDR randomization does not involve the cysteine residue at position L34 in the CDR-L1 according to the Kabat numbering scheme because otherwise the formation of the stabilizing interdomain disulfide bridge would not be enabled. For CDR-L3, the number of randomized amino acid position is typically at least two, if any. For CDR-L2, the number of randomized amino acid positions is typically one or more, if any.

The CDR regions of the present ds-scFvs may be embedded in any appropriate framework. At least for therapeutic applications, the framework is preferably a humanized framework or a framework derived from a human antibody. In some embodiments, the framework is that of Trastuzumab (also known as hu4D5), which has been successfully used in various CDR grafting studies. The amino acid sequence of Trastuzumab's framework is set forth in SEQ ID Nos. 1-8, such that SEQ ID NO:1 represents FR-H1, SEQ ID NO:2 represents FR-H2, SEQ ID NO:3 represents FR-H3, SEQ ID NO:4 represents FR-H4, SEQ ID NO:5 represents FR-L1, SEQ ID NO:6 represents FR-L2, SEQ ID NO:7 represents FR-L3, while SEQ ID NO:8 represents FR-L4. Therefore, in some embodiments, the ds-scFv of the invention comprises a V_{H} domain comprising framework areas of SEQ ID NO:1, SEQ ID NO:2, SEQ ID NO:3 and SEQ ID NO:4 in this order, and a V_{L} domain comprising framework areas of SEQ ID NO:5, SEQ ID NO:6, SEQ ID NO:7 and SEQ ID NO:8 in this order. In the V_{H} domain, cysteine residues forming the native intradomain disulfide bridges are located in FR-H1 and FR-H3, more specifically at the position 22 in SEQ ID NO: 1, and at the position 30 of SEQ ID NO:3, corresponding to positions H22 and H92 according to the Kabat numbering scheme in the ds-scFv, respectively. In the V_{L} domain, cysteine residues forming the native intradomain disulfide bridges are located in FR-L1 and FR-L3, more specifically at the position 23 in SEQ ID NO: 5, and at the position 32 of SEQ ID NO:7, corresponding to positions L23 and L88 according to the Kabat numbering scheme in the ds-scFv, respectively. In accordance with some aspects of the invention, namely ds-scFvs lacking the other native intradomain disulfide bridge, the framework has been engineered not to contain one or both of the above-mentioned native intradomain-disulfide-bridge-forming cysteine residues for either the V_{H} or the V_{L}.

However, the framework need not be 100% identical to the above-disclosed sequences but may vary, provided that functional or structure properties of the ds-scFvs remain essentially unaltered. Thus, in some embodiments, one or more of the framework regions may be functionally equivalent conservative sequence variants of the sequences set forth above, or may have at least 80%, 85%, 90%, 91%, 92%, 93%, 94%, 95%, 96%, 97%, 98%, or 99% sequence identity with the sequences set forth above. Moreover, the framework may be based on, or essentially correspond to, any framework encoded by a V and a J gene segment. Consensus human framework regions can also be employed, for example as described in U.S. Pat. No. 6,300,064.

Especially when the framework of Trastuzumab, or a functionally equivalent conservative sequence variant of said framework, is employed, the tryptophan H103 according to the Kabat numbering scheme used for defining the position of the artificially introduced cysteine residue in the CDR-H3 loop corresponds to the first amino acid of FR-H4, i.e., tryptophan at amino acid position 1 of SEQ ID NO:4.

In some embodiments employing the framework of Trastuzumab, the ds-scFv of the invention may comprise a V_{H} set forth in SEQ ID NO:48 and a V_{L} set forth in SEQ ID NO:49 in either order, intervened by a peptide linker. In such embodiments, the CDR loops can have varying amino acid compositions and lengths depending on their target antigens, the length of CDR-H1 being 5 amino acids, the length of CDR-H2 being 16-19 amino acids, preferably 17 amino acids, the length of CDR-H3 being 12-21 amino acids, preferably 13 amino acids, the length of CDR-L1 being 11 amino acids, the length of CDR-L2 being 7 amino acids and the length of CDR-L3 being 9-11 amino acids, preferably 9 amino acids. The fourth last amino acid in the CDR-H3 (i.e., the amino acid residue at position −4 counted from the first amino acid of FR-L4) must be cysteine. Also the last amino acid of CDR-L1 (i.e., the amino acid residue at position −1 counted from the first amino acid of FR-L2) must be cysteine. Functionally equivalent conservative sequence variants of the above-disclosed ds-scFvs are also encompassed by the present invention.

In those embodiments in which the CDR-H3 is composed of 13 amino acids, the amino acid position −4 counted from tryptophan H103 corresponds to the amino acid position H100B according to the Kabat numbering scheme. In some embodiments, cysteine residues forming the artificially disulfide bridge are at positions H100B and L34 according to the Kabat numbering scheme.

It should be understood that the ds-scFvs of the invention can be non-human (e.g., mouse, rabbit, goat), chimeric, humanized (non-human CDRs integrated into humanized framework regions) or fully human (both the framework and the CDRs derived from a human antibody). However, humanized and fully human ds-scFvs are preferred especially for therapeutic purposes. For diagnostic and some other non-therapeutic purposes, the ds-scFvs need not be humanized or fully human, but may equally be non-human or chimeric ds-scFvs. Notably, the source and the type of the V_{H} can differ from those of the V_{L}.

The V_{H} and V_{L} domains in the present ds-scFvs are connected by a flexible linker peptide that is usually 15-20 amino acids long. However, in some embodiments, the linker may be 12-15 amino acids long or even shorter, while in some other embodiments, the linker may be longer than 20 amino acids, such as 25 or even 30 amino acids. The linker keeps the C-terminus of one variable domain and the N-terminus of the other domain at a distance that favors proper folding and formation of the antigen-binding site while also minimizing oligomerization of the ds-scFv. It is generally believed that short linkers (typically 12-15 amino acids or less) will prevent the physical association of the two V domains within the same polypeptide and lead to the formation of multimers, while long linkers (typically longer than 20 amino acids) may favor proteolysis or weak domain association in scFvs.

In some embodiments, multimers of the present ds-scFvs may be desired. For example, it is envisaged that the present ds-scFvs may be utilized in generating diabodies, i.e., bispecific antibodies composed of two scFvs with different antigen-binding specificities, one or both of them being ds-scFvs of the invention. In such embodiments, the linker peptide should be short, usually 5-10 amino acids, such that neither of the scFv chains can form a functional scFv on their own, thereby inducing formation of diabodies composed of different scFv partners. Linkers of only 1-4 amino acids result predominantly in formation of trimeric and tetrameric constructs.

Usually in ds-scFvs, the linker peptide is composed of mainly glycine and serine residues. The 15-amino acid (glycine₄-serine)₃ peptide linker set forth in SEQ ID NO:9, also expressed as (Gly₄-Ser)₃ or (GGGGS)₃ linker, is most widely used. However, longer linkers, such as 20-amino acid (Gly₄Ser)₄ or (GGGGS)₄ set forth in SEQ ID NO: 10 may be used, for example, to minimize oligomerization. In some embodiments, other residues, such as charged residues glutamate (Glu) and/or lysine (Lys), can also be incorporated into the linker, for example to enhance solubility. In some embodiments, amino acid residues such as alanine (Ala) and/or threonine (Thr) may be incorporated into the linker as for example in the case of linker GGGGSGAGGSGGGGTGGGGS (SEQ ID NO: 11) used in the present examples.

The order of the V_{H} and V_{L} domains in the present ds-scFvs, also called orientation, can be either V_{L}-linker-V_{H} (LH, also expressed as V_{L}-V_{H}) or V_{H}-linker-V_{L} (HL, also expressed as V_{H}-V_{L}). However, individual scFvs may perform better in one configuration than in the other, for example in terms in their binding properties. Moreover, in some embodiments, expression yields may vary depending on the configuration, such that the HL orientation is usually preferred.

In some embodiments, ds-scFvs of the invention may comprise one or more additional peptide tags for various purposes, such as to facilitate purification, isolation, immobilization and/or detection. Various peptide tags suitable for such and other purposes are readily available in the art. Non-limiting examples of such peptide tags are set forth later in this description.

Stabilized ds-scFv molecules of the invention have improved stability as compared to corresponding conventional scFv molecules. Protein stability is usually measured by the reversible unfolding of the protein with either heat or a chaotrope such as guanidine hydrochloride or urea, using methods known in the art. Accordingly, in some embodiments, the measure of protein stability is thermostability, i.e., resistance to irreversible unfolding by thermal challenge. In some embodiments, the measure of protein stability is pH dependent, i.e., resistance to protein unfolding by pH variations. Moreover, the measure of protein stability may also be tolerance to proteases. Especially for antibodies, stability may also be measured as stability in serum.

Thermostability can be measured using a number of non-limiting biophysical or biochemical techniques known in the art. Perhaps the most common method for measuring protein thermal shifts is differential scanning fluorimetry (DSF) or thermofluor, which takes advantage of small fluorescent molecules whose fluorescence is enhanced when bound to exposed hydrophobic surfaces such as those created by protein unfolding. Alternatively, thermostability can be determined by other analytical techniques, such as differential scanning calorimetry (DSC) and temperature dependent circular dichroism spectroscopy (CD). All these biophysical techniques allow for the determination of thermal unfolding transitions. The temperature at which the protein unfolds is indicative of overall protein stability.

In other embodiments, thermostability can be measured biochemically. An exemplary biochemical method for assessing thermostability is a thermal challenge assay, wherein a composition whose thermostability is to be determined is exposed to a range of elevated temperatures for a set period of time. For example, in some embodiments, one or more test compositions (e.g., ds-scFv of the invention and/or reference scFv molecules, or particles of an expression library displaying them) are subjected to a range of increasing temperatures, e.g., for about 1 to about 1.5 hours. The activity of the test composition is then assayed by a relevant biochemical assay. Preferably, the assay is a binding assay, employed to determine any change in the target molecule-binding properties of the heat-challenged composition. The binding assay may be, for example, a functional or quantitative ELISA assay. The temperature at which the antigen-binding property is lost, is indicative of overall thermostability.

In some embodiments, thermostability can be evaluated by measuring the melting temperature (Tₘ) of a test composition using any of the above-mentioned techniques. The melting temperature is the temperature at the midpoint of a thermal transition curve wherein 50% of molecules of a composition become denatured, as determined e.g., by unfolding or loss of antigen binding. Tₘ is considered to be the standard parameter for describing the thermostability of a protein.

In some embodiments, the ds-scFvs of the invention have a thermostability that is greater than about 1, about 1.25, about 1.5, about 1.75, about 2, about 3, about 4, about 5, about 6, about 7, about 8, about 9, about 10 or more degrees Celsius than that of a control molecule, such as a corresponding conventional scFv molecule.

Surprisingly, the present interdomain disulfide bridge between CDR-H3 and CDR-L1 loops increased the thermostability of the present ds-scFvs markedly better than H44-L100, a disulfide bridge suggested earlier to serve as a universal location for scFv stabilization.

Ds-scFvs of the invention can be used as such for various research, diagnostic and therapeutic purposes, largely depending on their antigen-binding specificities. In addition, they may be utilized as building blocks for various molecular entities, including but not limited to, engineered therapeutic proteins, such as bispecific antibodies and CAR T-cells.

Bispecific T-cell engagers (BiTEs) are a class of artificial bispecific monoclonal antibodies that contain two scFvs against different antigens. The other scFv targets a protein complex cluster of differentiation (CD3) on T-cells whereas the other scFv targets a disease-specific antigen, thereby forming a link between the T-cell and the diseased cell, such as a tumor cell. In the close vicinity to the diseased cells, the T-cells destroy them by programming them to undergo apoptosis. It is envisaged that ds-scFvs of the invention are suitable as building blocks for constructing BiTes.

It is also envisaged that the ds-scFv of the invention can be utilized to form various other types of bivalent or bi- or multi-specific antibody constructs. For example, it can be expressed as fused to either N-terminus or C-terminus of either the light or heavy chain of a full-length antibody construct. Alternatively, one or both Fab arm(s) of a bispecific antibody can be replaced with the ds-scFv of the invention while maintaining the Fc part (i.e. fragment crystallizable) for immunomodulatory properties. This results in an antibody construct that is significantly smaller than an intact IgG antibody, but still expected to have essentially unaltered immunomodulatory properties and long half-life time due to the presence of the Fc part. Accordingly, ds-scFvs of the invention are suitable as building blocks for constructs comprising a fusion of the ds-scFv and an Fc part of an antibody. Further bispecific constructs can be created by fusing the ds-scFv of the invention to alternative protein scaffold-based affinity reagents such as Design Ankyrin Proteins (DARPins), nanobodies, affibodies, and the like. Such binder molecules are readily available in the art.

It is further envisaged that ds-scFvs of the invention may also be utilized in CAR T-cell therapies. CAR T-cell therapy is a type of cancer immunotherapy that uses a patient's T-cells to find and kill tumor cells. CAR T-cells are engineered T-cells that express an artificial T-cell receptor, CAR, on the cell surface. These artificial T-cell receptors are chimeric—they contain both antigen-binding and T-cell activating domains. In addition to the antigen-binding domain (ds-scFV of the invention) and T-cell activating domain, CAR consists of hinge, transmembrane, and co-stimulatory domains as is well known to those skilled in the art. When a CAR has an ds-scFv that binds cancer-related antigens, binding of the ds-scFv part of the receptor can activate T cells to kill cancer cells.

In some further embodiments, ds-scFvs of the invention may be comprised in molecular entities such as pharmaceutically active agents, drugs, radioisotopes, enzymes (e.g., alkaline phosphatase) or chelating agents, to name some non-limiting examples. Depending on the type of the molecular entity, the given ds-scFv may be either conjugated or recombinantly fused to said molecular entity using means and methods readily available in the art.

The present invention also encompasses a nucleic acid molecule encoding the ds-scFv of the invention and its various embodiments.

Turning now to a particular embodiment, the present invention provides anti-HER2 ds-scFv variants. They were derived from an scFv variant of Trastuzumab by introducing the stabilizing interdomain disulfide bridge of the invention to a novel position, namely between CDR-H3 and CDR-L1, more specifically between position −4 counted from H103 and position L34 according to the Kabat numbering scheme. In some embodiments, the position −4 counted from H103 is the position H100B.

Surprisingly, introduction of the interdomain disulfide bridge of the invention increased the thermostability of ds-scFvs displayed on phages and the soluble ds-scFv proteins by 10° C. compared to that of the original scFv not having the interdomain disulfide bridge. Importantly, the interdomain disulfide bridge at position H44-L100 according to the Kabat numbering scheme had a significantly lower effect on the thermostability than the interloop disulfide bridge of the invention. The Tₘ values for comparative ds-scFvs with the H44-L100 modification were only 3-4° C. higher than those of the original scFv but 7° C. lower than those of the variants with the an interloop disulfide bridge in the novel H100B-L34 position examined in this study.

Moreover, anti-HER2 scFvs with the added interdomain disulfide bridge retained the antigen-binding properties of their parent scFv in both orientations.

In some embodiments, the anti-HER2 ds-scFv comprises essentially same CDR sequences as Trastuzumab, except for the cysteine residues that form the interdomain disulfide bridge of the invention. Thus, in some embodiments, the anti-HER2 ds-scFv comprises CDR-H1 having an amino acid sequence of SEQ ID NO:12, the CDR-H2 having an amino acid sequence of SEQ ID NO:13, the CDR-H3 having an amino acid sequence of SEQ ID NO:14, the CDR-L1 having an amino acid sequence of SEQ ID NO:15, the CDR-L2 having an amino acid sequence of SEQ ID NO:16 and the CDR-L3 having an amino acid sequence of SEQ ID NO:17, or a conservative sequence variant of said CDR sequences provided that the HER2-binding properties of the anti-HER2 ds-scFv is not lost. For comparison, CDR-H3 and CDR-L1 of Trastuzumab are shown in SEQ ID NO:18 and SEQ ID NO:19, respectively. The cysteine residues forming the artificial interdomain disulfide bridge are at position 10 in SEQ ID NO:14 and at position 11 in SEQ ID NO:15.

Thus, in some embodiments, the anti-HER2 ds-scFv of the invention comprises a V_{H} domain comprising CDR sequences SEQ ID NO:12, SEQ ID NO:13 and SEQ ID NO:14, and a V_{L} domain comprising CDR sequences SEQ ID NO:15, SEQ ID NO:16 and SEQ ID NO:17, the V_{H} and V_{L} domains being in either orientation. In some further embodiments, such V_{H} and V_{L} domains are intervened by a peptide linker, for example a peptide linker selected from peptide linkers of SEQ ID NO:9, SEQ ID NO:10, SEQ ID NO:11 and functionally equivalent sequence variants thereof disclosed above.

In some further embodiments, the anti-HER2 ds-scFv of the invention comprises a V_{H} domain comprising or consisting of an amino acid sequence set forth in SEQ ID NO:31, and a V_{L} domain comprising or consisting of an amino acid sequence set forth in SEQ ID NO:32, in either orientation. The cysteine residues forming the artificial interdomain disulfide bridge are at positions 106 in SEQ ID NO:31 and at 34 in SEQ ID NO:32; whereas the cysteines forming the native intradomain disulfide bridges are at positions 22 and 96 in SEQ ID NO:31 and at positions 23 and 88 in SEQ ID NO:32. In some further embodiments, such V_{H} and V_{L} domains are intervened by a peptide linker, for example a peptide linker selected from peptide linkers of SEQ ID NO:9, SEQ ID NO:10, SEQ ID NO:11 and functionally equivalent sequence variants thereof disclosed above.

In some still further embodiments, the anti-HER2 ds-scFv is in HL orientation and has an amino acid sequence of SEQ ID NO:20. In some even further embodiments, the anti-HER2 ds-scFv is in LH orientation and has an amino acid sequence of SEQ ID NO:21. In these embodiments, the cysteine residues forming the artificial interdomain disulfide bridge are at positions 106 and 174 in SEQ ID NO:20 and at positions 34 and 235 in SEQ ID NO:21. The cysteine residues forming the intradomain disulfide bridges are positions 22, 96, 163, 228 in SEQ ID NO:20, and at positions 23, 88, 151, 225 in SEQ ID NO:21. The DNA sequence coding for SEQ ID NO:20 is set forth in SEQ ID NO:45, whereas the DNA sequence coding for SEQ ID NO:21 is set forth in SEQ ID NO:46.

In some embodiments, CDR-H3 of the anti-HER2 ds-scFv may contain one or more amino acid alterations (substitutions, deletions and/or insertions) between H93-H100A according to the Kabat numbering scheme (corresponding to amino acid residues in 1-9 in SEQ ID NO:14, amino acid residues 97-105 in SEQ ID NO:31, amino acid residues 97-105 in SEQ ID NO:20 or amino acid residues 226-234 in SEQ ID NO:21), provided that the anti-HER2 ds-scFv still shows specific binding to HER2, preferably at Kd<10 nM. In other words, said amino acid alterations in the CDR-H3 are located upstream from the cysteine at the residue −4 counted from conserved tryptophan H103 of the FR-H4. These amino acid alterations are preferably located at positions corresponding to amino acid residues 3-9 in SEQ ID NO:14, amino acid residues 99-105 in SEQ ID NO:31, amino acid residues 99-105 in SEQ ID NO:20 or amino acid residues 228-234 in SEQ ID NO:21).

Alternatively or additionally, the anti-HER2 ds-scFvs of the invention may contain one or more amino acid alterations (substitutions, deletions and/or insertions) also in the CDR-H1 loop and/or in the CDR-H2 loop as compared to the amino acid sequences disclosed herein.

Independently from the presence or absence of amino acid alterations in the CDR-H1, CDR-H2 and/or CDR-H3, the anti-HER2 ds-scFvs of the invention may in some embodiments contain one or more amino acid alterations (substitutions, deletions and/or insertions) in the CDR-L1 and/or CDR-L3 as compared to the amino acid sequences disclosed herein. In some embodiments, also the CDR-L2 loop may contain one or more amino acid alterations (substitutions, deletions and/or insertions) as compared to the amino acid sequence of the CDR-L2 loop disclosed herein.

As readily understood by those skilled in the art, a requirement for the above-mentioned amino acid alterations is that the anti-HER2 ds-scFvs still show specific binding to HER2, preferably at Kd<10 nM. The presence or absence of said binding specificity can be easily determined by means and methods available in the art. Usually, the order of preference for diversifying the CDR loops is CDR-H3, either one or both of CDR-H1 and CDR-H2, either one or both of CDR-L1 and CDR-L3, and CDR-L2. To facilitate said diversification, one of the two intradomain disulfide bridges may be abolished by replacing one or both of the cysteine residues normally participating in the bridge formation, including those specifically mentioned with respect to given SEQ ID NOs, in accordance with what is stated elsewhere in this description. Said intradomain disulfide bridge may be reintroduced later which is also in accordance with what is stated elsewhere in the description.

In some embodiments, the anti-HER2 ds-scFvs of the invention are provided for use in treating cancer, especially breast cancer or stomach cancer. In some further embodiments, they may be used for bispecific antibodies, BiTEs or CAR T-cell therapies.

Notably, stabilizing interdomain disulfide bridge-forming cysteine residues of the invention may be engineered into any existing scFvs by techniques well known in the art, including for example site-directed mutagenesis. In vitro display technologies may also be applied to generate ds-scFvs of the invention.

Thus, an aspect of the present invention relates to a recombinant library of particles, the library displaying a plurality of present ds-scFvs against various antigens, such as HER2. Further aspects relate to a method of constructing such libraries and uses thereof to obtain novel ds-scFvs with desired antigen-biding properties. Accordingly, ds-scFvs towards a desired antigen can be easily generated from the present library by antigen screening without the need of host animal immunizations and hybridoma production, thereby substantially shortening the time and efforts generally required for the production of an antibody via a conventional manner.

Accordingly, the present invention provides a method of constructing a recombinant expression library, more specifically a library of particles that display ds-scFvs of the invention on them, thereby linking genotypes and phenotypes of the particles. The particles forming the library may be phage particles, ribosomes, yeast cells, bacterial cells or mammalian cells. In some preferred embodiments, the library is a phage display library, while some other embodiments employ both phage display and mammalian cell display libraries.

To construct a display library of the invention, codons for stabilizing interdomain disulfide bridge-forming cysteine residues are introduced into appropriate positions in nucleic acids encoding V_{H} and V_{L} with desired diversity using methods well known in the art. Preferably, the nucleic acids are DNA molecules. The diversity may originate from natural or non-natural sources. For example, the diversity may originate from immune libraries constructed from the variable domain genes isolated from the B-cells derived from immunized animals or humans. Alternatively, the diversity may originate from naïve libraries can be derived from the variable domain genes isolated from nonimmunized donors. In some further embodiments, the diversity may originate from fully synthetic libraries usually derived from nonimmune sources and subjected to computational in silico design and gene synthesis. In the synthetic libraries, humanized frameworks or frameworks derived from human antibodies are combined with CDRs randomized at positions most likely to contribute to antigen recognition and binding. In semisynthetic libraries, the natural and in silico designed CDR diversity is combined with or without CDR randomization. CDR randomization may be achieved, for example, by PCR-based assembly of synthetic oligos, as is well known in the art.

Owing to its important role in antigen recognition and binding, the present display library of ds-scFvs is in some preferred embodiments a CDR-H3 library. Such a library may be constructed by diversifying the CDR-H3 loop length and/or amino acid composition of an existing antibody or an antigen-binding fragment thereof. In some embodiments, the length of the CDR-H3 loop is preferably from 12 to 21 amino acids.

Also the other CDR loops may vary in length. In some embodiments, and especially in those that employ the framework of Trastuzumab or a functionally equivalent conservative sequence variant thereof, CDR-H1 is usually 5 amino acids long, CDR-H2 is usually 16-19, preferably 17 amino acids long, whereas CDR-H3 is usually 12-21 amino acids long. In some embodiments, CDR-H3 is 13 amino acids long. CDR-L1, in turn, is usually 11 amino acids long, CDR-L2 is usually 7 amino acids long, whereas CDR-L3 is usually 9-11 amino acids long. In some embodiments, CDR-L3 is 9 amino acids long. In some embodiments, one or more of the CDR loops may contain one or more randomized amino acids in accordance with what is stated elsewhere in this description.

Since the length of CDR-H3 may vary, the amino acid position of the cysteine residue participating in the artificial disulfide bridge must be defined indirectly through its location with respect to the highly conserved tryptophan residue at position 1 of FR-H4. This definition applies regardless of the framework employed. In some embodiments, the recombinant expression library is based on variants of V_{H} set forth in SEQ ID NO:48 and variants of V_{L} set forth in SEQ ID NO:49. More generally, the recombinant expression library is in some embodiments based on V_{H} comprising framework regions set forth in SEQ ID NOs:1-4 and variants of V_{L} comprising frame work regions set forth in SEQ ID NOs:5-8. In accordance with what is stated above, said sequences may be engineered such that either one or both of amino residues 22 of SEQ ID NO:1 and 30 of SEQ ID NO:3 are not cysteines or one or both of amino acid residues 23 of SEQ ID NO:5 and 32 of SEQ ID NO:7 are not cysteines thereby preventing the formation of one of the two native intradomain disulfide bridges. With respect to sequences SEQ ID NO:48 and SEQ ID NO:49 this means that they may be engineered such that either one or both of the amino acid residues at positions 22 and 98 of SEQ ID NO:48 are not cysteines or such that either one or both of the amino acid residues at positions 23 and 88 of SEQ ID NO:49 are not cysteines, thereby preventing the formation of one of the two native intradomain disulfide bridges.

Like in the context of ds-scFvs above, any appropriate framework can be employed for creating a recombinant expression library of the invention. Preferably, the framework is a humanized framework or a framework derived from a human antibody. In some specific embodiments, the framework is that of Trastuzumab or a conservative or other variant thereof, as is set forth above. In some other embodiments, the framework is a human consensus framework.

Depending on the source of the CDRs and/or the framework, ds-scFvs displayed by the recombinant expression library of the invention may be denoted as recombinant non-human, chimeric, humanized, fully human or artificial (in silico designed) as understood by those skilled in the art. Notably, the source and design of the V_{H} can differ from those of the V_{L}.

Once the nucleic acid molecules encoding V_{H} and V_{L} originating from the desired diversity and comprising the stabilizing interdomain disulfide bridge-forming cysteine residues at CDR-H3 and CDR-L1, preferably at the position −4 counted from H103 in the FR-H4 and at the position L34 in the CDR-L1 according to the Kabat numbering scheme and preferably lacking one or both of the native cysteines participating in the intradomain disulfide bridge formation within either V_{H} or V_{L}, have been engineered, they are cloned into an expression vector to create a library of ds-scFvs. The choice of the expression vector dependents on the type of a display library to be created, as is readily understood by those skilled in the art. A wide variety of suitable expression vectors are commercially available.

For phage display, nucleic acids coding for an ds-scFv are fused to the phage coat protein gene in a phage or phagemid vector, causing the phage to display the scFv on its surface. The most widely employed phage display format utilizes filamentous bacteriophage M13 and fuses the protein of interest into the phage coat protein pIII enabling high-frequency monovalent display. In this approach, phagemid vectors with randomly cloned nucleic acids coding for a plethora of different ds-scFv are electroporated into E. coli followed by infection with a helper phage to produce a phage library, each phage displaying a different ds-scFvs on its surface. The art is replete with suitable vectors, helper phages as well as other means for constructing a phage display library.

For mammalian cell display, nucleic acids coding for an ds-scFv with a membrane anchor segment may be integrated into the genome of mammalian cells, causing the cells to display the ds-scFv on their surface. Various mammalian cell lines can be used for this purpose including, but not limited to, CHO (Chinese hamster ovary) and HEK293 (Human embryonic kidney) cells. There are various strategies to incorporate the ds-scFv expression construct into the genome of the cell; these include e.g., the use of homologous recombination and a landing pad targeting site-specific recombinase. The efficiency of homologous recombination can be increased, for example, using DNA double-strand breaks inducing zing-finger or TALE-effector nucleases or CRISPR/Cas system. The cells displaying ds-scFvs with certain binding specificities can be enriched and isolated by antigen-coated magnetic bead-based separation or FACS (fluorescence-activated cell sorting)-based screening using a fluorescent labelled antigen, or by any other technique suitable for this purpose.

Once a recombinant expression library, such as a phage display library, has been generated, it may be used to select ds-scFvs with desired antigen-binding properties by a process called panning, typically involving several rounds of selection against one or more target antigens, preferably immobilized on a solid surface (e.g., beads or microtiter plates). After incubation with the target antigen, non-reacting phages are removed, for example by extensive washing. Bound phages, in turn, are eluted and enriched by amplification in appropriate host cells (typically bacterial cells) prior to the following selection round. These steps are typically repeated two to four times to achieve the most specific phages against the desired target antigen. In some embodiments, the stringency of the selections may be increased for each selection round to enrich for ds-scFvs with high affinity and specificity.

In some embodiments, the panning process may also involve one or more rounds of negative selection. To this end, negative selection proteins, i.e., non-target antigens, are contacted with the phage display library. Phages that bind to the non-target antigens are removed and the remaining phage stock is used for positive selection against the target antigen.

After the panning rounds, it is usually tested whether antigen-specific phage binders have been enriched, typically by using an appropriate immunoassay. Individual clones are then screened from the enriched pools of antigen-specific phage binders. The positive clones may then be further characterized, for example, by determining their antigen-binding characteristics and/or by sequencing.

Generally, phage display libraries allow greater diversity than display libraries of mammalian cells. In other words, the repertoire of different variants in phage display libraries may be significantly larger than in display libraries of mammalian cells. Therefore, the former libraries are sometimes preferred over the latter.

On the other hand, the presence of three disulfide bridges, namely one interdomain disulfide bridge of the invention and two naturally occurring intradomain disulfide bridges, in the ds-scFvs to be displayed may lower the display rate (i.e., yield) on phage surfaces. It is envisaged that the display efficiency is not significantly reduced in mammalian cell display libraries, since mammalian cells can well express molecules with three disulfide bridges.

It has now been surprisingly realized that benefits of phage display and mammalian cell display libraries can be combined. To this end, a phage display library of ds-scFv variants with the interdomain disulfide bridge of the invention and one naturally occurring intradomain disulfide bridge only (instead of two intradomain sulfide bridges native to scFvs, located in V_{L} between cysteines at positions L23 and L88, and in V_{H} between positions H22 and H92 according to the Kabat numbering scheme) is first created. This enables creating a display library with very high diversity without compromising the display efficiency, since phages are capable of expressing molecules with two disulfide bridges at high rates. The missing intradomain disulfide bridge may be engineered to be lacking either from the V_{H} or V_{L}. For the former option, the V_{H} may in some embodiments have an amino acid sequence set forth in SEQ ID NO:34; whereas for the latter option, the V_{L} may in some embodiments have an amino acid sequence set forth in SEQ ID NO:33. In some preferred embodiments, the intradomain disulfide bridge is missing from the second V domain, i.e., from the V_{H} domain when the ds-scFv is in the LH orientation, and from the V_{L} domain when the ds-scFv is in the HL orientation. In some embodiments, the latter option (HL orientation) is preferred.

Once the most promising ds-scFvs have been obtained from a large phage display library, the missing intradomain disulfide bridge-forming cysteines may be reintroduced by employing V_{L} shuffling (if the intradomain disulfide bridge is missing from the V_{L}) or by V_{H} shuffling (if the intradomain disulfide bridge is missing from the V_{H}). Mammalian cell display technique may then be employed to subject a library of ds-scFvs containing all three disulfide bridges, namely the non-natural interdomain disulfide bridge of the invention and the two natural intradomain disulfide bridges, so obtained to selection with the target antigen. This approach enhances the success rate of obtaining proper ds-scFvs specific to the target antigen, since larger diversities of phage display libraries can be exploited despite their lower display efficiency.

Accordingly, in an embodiment of the invention, high diversity is first introduced into the CDRs of V_{H}, while V_{L} is not diversified or is diversified to a limited extent, comprising for example >1E1>1E2>1E3>1E4>1E5 variants. The first library so created may contain, for example, >1E8 or >1E9 or >1E10 V_{H} variants. In some embodiments, the orientation of ds-scFvs in such a library is V_{H}-V_{L}, the V_{L} lacking the native intradomain disulfide bridge. In some further embodiments, such a library is then expressed on filamentous phages and selected against a target antigen.

In some embodiments, V_{L} shuffling may then be employed to introduce additional diversity into the library. In other words, antigen specific V_{H} domains enriched from the phage display library may be combined with diversified V_{L} domains that contain the native intradomain disulfide bridge. The resulting second library may then be subjected to antigen specific selection using mammalian cell display technique.

It is to be noted that in the approach described above by means of preferred embodiments, other cell-based display techniques, such as yeast display, may be employed instead of mammalian cell display, although mammalian cell display is generally preferred. Likewise, V_{L}-V_{H} orientation may be used instead of V_{H}-V_{L} orientation. In such cases, the native intradomain disulfide bridge is missing from the V_{H}, and the first library has high diversity in the CDRs of V_{L}, while V_{H} is not diversified or is diversified to a limited extent. V_{H} shuffling may then be employed to introduce additional diversity into the library, while also reintroducing the native intradomain disulfide bridge into the V_{H}, followed by subjecting the second library so created to target antigen specific selection using a cell-based display technique.

In some embodiments, further diversity may be introduced, preferably into the first library described above, by mutagenesis techniques readily available in the art, such as random mutagenesis, for example by error prone PCR. In some embodiments, mutagenesis may be employed instead of or in addition to V_{L} or V_{H} shuffling.

Moreover, diversity may also be introduced into the first and/or the second library, preferably into the first library, by CDR randomization using means and methods readily available in the art, in accordance with what is stated elsewhere in this description, although not repeated herein.

It is to be understood that although it may be beneficial to combine phage display and mammalian cell display approaches as explained above, target-specific ds-scFvs of the invention containing all three disulfide bridges may also be successfully obtained either from phage display libraries (despite a lower yield) or mammalian cell libraries (despite lower diversity).

Each display particle carries genetic information for the recombinant polypeptide that it displays on its surface. This feature allows for identifying nucleic acids encoding an ds-scFv exhibiting desired specificity by selecting that particle which carries it from a potentially very complex recombinant library. Nucleic acids from the best clones may then be isolated, inserted into a suitable expression vector, and transfected or transformed into a compatible expression host to produce the ds-scFv according to standard recombinant technology.

Numerous types of suitable expression vectors are available and include, but are not limited to, plasmids and modified viruses which are maintained in the host cell as autonomous DNA molecules or integrated in genomic DNA. The vector system must be compatible with the host cell employed as is well known in the art Preferably, DNA encoding an ds-scFv of the present invention is operably linked to one or more heterologous expression control sequences permitting expression of the ds-scFv. Suitable control sequences are readily available in the art and include, but are not limited to, promoter, leader, polyadenylation, and signal sequences.

Expression vectors may be transfected or transformed into host cells by standard techniques commonly used for the introduction of exogenous nucleic acids into a prokaryotic or eukaryotic host cell including, but not limited to, electroporation, nucleofection, sonoporation, magnetofection, heat shock, calcium-phosphate precipitation, DEAE-dextran transfection and the like.

Ds-scFvs can be expressed in various expression systems including but not limited to prokaryotic host cells such as bacteria (e.g., E. coli, bacilli), yeast (e.g., Pichia pastoris, Saccharomyces cerevisiae), and fungi (e.g., filamentous fungi), as well as eukaryotic hosts such as plant cells, insect cells (e.g., Sf9) and mammalian cells (e.g., CHO cells). Host cells transfected with an expression vector comprising nucleic acid, preferably DNA, coding for an ds-scFv of the invention are to be cultured under conditions suitable for the production of the ds-scFv followed by recovering the ds-scFv obtained. Ds-scFvs of the invention may also be produced by in vitro protein expression according to protocols known in the art.

Bacterial cell expression systems, such as E. coli, are the most rapid and inexpensive form and thus in some instances preferred for the production of ds-scFvs. In such embodiments, the ds-scFvs are preferably targeted to their oxidizing periplasmic space that contain chaperones and disulfide isomerases that enable correct folding of the protein and the formation of disulfide bridges, respectively. The secretion can be directed to the periplasmic space with the assistance of signal peptides (SP), such as pelB, that are linked to the N-terminal end of the ds-scFvs using standard recombinant techniques. Non-limiting examples of suitable signal peptides include the ones set forth in SEQ ID NO:22, SEQ ID NO:23, SEQ ID NO:25 and SEQ ID NO:28. The ds-scFvs can also be expressed in the cytoplasm of E. coli or other bacterial cells in high yields. However, due to the reducing environment of the cytoplasm, disulfide bonds cannot be formed in the majority of prokaryotic organisms. Therefore, ds-scFvs must be recovered from inclusion bodies into which they accumulate and refolded, which can be time-consuming and inefficient. There are, however, some exceptions, such as Origami™ B host strains and the like. Moreover, soluble expression of disulfide bonded proteins in the cytoplasm of bacterial cells, such as E. coli, is possible with a system known as CyDisCo, which is based on co-expression of a protein of interest along with a sulfhydryl oxidase and a disulfide bond isomerase.

In some embodiments, it may be desirable to express an ds-scFv of the invention as a fusion to one or more peptide or small protein tags that facilitate purification, isolation, immobilization and/or detection. Non-limiting examples of suitable affinity tags for purification or immobilization purposes include polyhistidine tags (His-tags), hemagglutinin tags (HAtags), glutathione-S— transferase tags (GST-tags), and biotin tags. Suitable detection tags include, but are not limited to, Myc-tag, FLAG-tag, fluorescent proteins, such as GFP, and enzyme tags that will generate a colored product upon contact with a chromogenic substrate. Non-limiting examples of suitable enzyme tags include alkaline phosphatase (AP) and (horseradish) hydrogen peroxidase (HRP). Also other tags such as biotin, avidin, and streptavidin may be employed for detection purposes. They can be detected with a biotin/avidin/streptavidin-binding protein that is conjugated to an enzyme, fluorophore or other reporter molecule. Vectors, other means, and methods for producing present ds-scFvs as fusion proteins are readily available in the art.

### EXAMPLES

### Example 1. Anti-HER2-Ds-scFv Constructs

Anti-HER2 scFv variants used in the examples were designed and genes were then ordered as cloned genes from Twist Bioscience (USA).

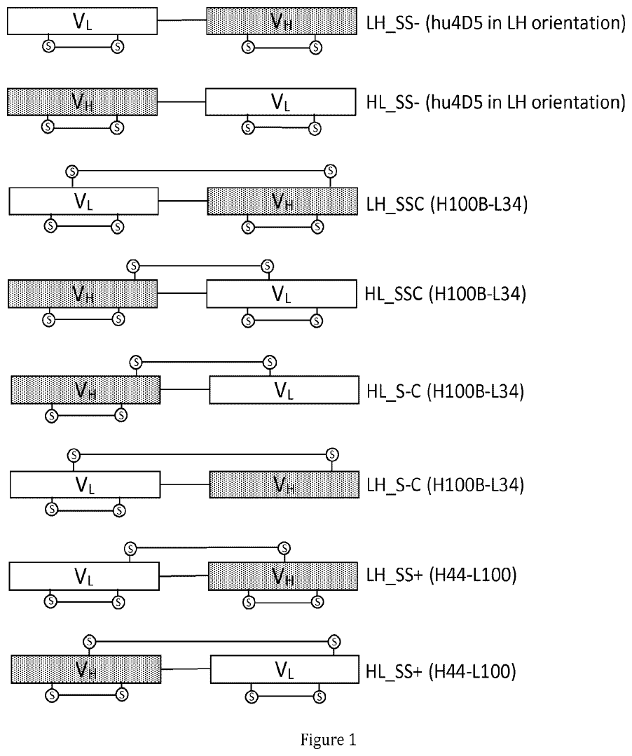

In the constructs an artificial disulfide bridge was introduced between CDR-H3 loop (position H100B according to Kabat numbering, corresponding to position 106 in SEQ ID NO:31) and CDR-L1 loop (position L34 according to Kabat numbering, corresponding to position 34 in SEQ ID NO:32) of the scFv fragment of trastuzumab (also known as hu4D5). In addition to the added interloop disulfide bridge, each of the V_{L} and V_{H} had a native intradomain disulfide bridge. In V_{L}, the intradomain disulfide bridge was between cysteines at positions L23 and L88 according to the Kabat numbering scheme (corresponding to positions 23 and 88 in SEQ ID NO:32, respectively). In V_{H}, the intradomain disulfide bridge was between cysteines at positions H22 and H92 according to the Kabat numbering scheme (corresponding to positions 22 and 96 in SEQ ID NO:31, respectively). ScFv was produced in both LH (V_{L}-V_{H}) and HL (V_{H}-V_{L}) orientations. Constructs having the all the three disulfide bridges (LH_SSC and HL_SSC, respectively) and constructs where the native intradomain disulfide bridges had been removed from the second domain in the scFv were tested. In LH_S-C intradomain disulfide bridge was removed from V_{H} (domain order V_{L}-V_{H}) and in HL_S-C from V_{L} (domain order V_{H}-V_{L}). The wild-type scFv having only the native intradomain disulfide bridges (LH_SS− and HL_SS−) was used as a control. In addition, a previously described construct containing a disulfide bridge between V_{L} and V_{H} in the position H44-L100 was expressed in both LH and HL orientations and evaluated as a reference. The schematic presentations of the constructs and the V_{L} and V_{H} amino acid sequences are presented in FIG. 1 and Table 1, respectively. All scFvs constructs contained a 20 aa glysine-serine peptide linker GGGGSGAGGSGGGGTGGGGS (SEQ ID NO:11) between the V_{L} and V_{H}.

Constructs used in Example 2 for expression of ds-ScFv in E. coli were designed to contain SfiI cloning sites at both ends. The ScFv genes were cloned at the SfiI sites in a periplasmic expression vector pAK400 (Krebber et. al., 1997) which contained pelB signal peptide (MKSLLPTAAAGLLLLAAQPAMA; SEQ ID NO:22), Lac promoter and resistance gene for chloramphenicol. His6-tag was introduced to the C-terminus of scFv from the vector.

Constructs used in Example 6 to study phage display were cloned at SfiI sited in phagemid vectors pEB32x (Huovinen et. al., 2013) and pEB3V3. pEB3V3 is identical to vector pEB32x but contains a modified pelB signal peptide MKYLLPTVVVGLLLLAAQPAMA (SEQ ID NO:23), encoded by atg aag tac ctt cta ccg acg gta gtc gtt gga ttg tta tta ctc gcg gcc cag ccg gcc atg gcg (SEQ ID NO: 24). pEB32x contained pelB signal peptide MKYLLPTAAAGLLLLAAQPAMA (SEQ ID NO:25), encoded by atg aaa tac cta ttg cct acg gca gcc gct gga ttg tta tta ctc gcg gcc cag ccg gcc atg gcg (SEQ ID NO:26)). From these vectors scFv is expressed as fused to the C-terminal domain of phage coat protein pIII. The vectors had Lac promoter, pelB signal peptide and chloramphenicol resistance gene for antibiotic selection.

The scFv construct used in Example 3 to expression of scFv in mammalian cells were designed to contain Kozak sequence gccgccacc and signal peptide MVLQTQVFISLLLWISGAYG (SEQ ID NO:28, Human Ig kappa chain V-IV region B17) at the N-terminal end as described by Vazquez-Lombardi et. al., 1997 and His6-tag at the C-terminus. The genes were ordered from Twist Bioscience cloned between the EcoRI and XbaI sites in expression Vector pTwistCMV Betglobin WPRE Neo (Twist Bioscience).

TABLE 1
Description of the scFv constructs evaluated
in the study and the mutations in their amino acid se-
quence in V_{H} and V_{L}. In scFv the V_{H} and V_{L} were joined
by a peptide linker GGGGSGAGGSGGGGTGGGGS (SEQ ID NO: 11) .

| | | V_{H} amino acid | V_{L} amino acid |
|---|---|---|---|
| | | sequence and | sequence and |
| Name | Description | mutations | mutations |
| LH_SS- | No interdomain | EVQLVESGG- | DIQMTQSPSSLSASV |
| (hu4D5) | disulfide bridge between | GLVQPGGSLRLS-CAASGENIK- | GDRVTITCRASQDVN |
| | the CDR-L1 and | DTYIHWVR- | TAVAWYQQKPGKAP- |
| | CDR-H3 | QAPGKGLEWVARI- | KLLIYSASFLYSGVP |
| | Expressed in | YPT- | SRFSGSRSGTDFT- |
| | the variable | NGYTRYADSVKGRFT | LTISSLQPEDFA- |
| | light chain | ISADTSKNTAYLQMN | TYYCQQHYT- |
| | (V_{L})-variable heavy | SLRAEDTAVYYCSRW | TPPTFGQGTKVEIKR |
| | chain (V_{H}) | GGDGFYAMDYWGQGT | T (SEQ ID |
| | orientation | LVTVSS (SEQ ID NO: 29) | NO: 30) |
| | (LH) | | |
| HL_SS- | No interdomain | EVQLVESGG- | DIQMTQSPSSLSASV |
| (hu4D5) | disulfide | GLVQPGGSLRLS- | GDRVTITCRASQDVN |
| | bridge between | CAASGFNIK- | TAVAWYQQKPGKAP- |
| | the CDR-L1 and | DTYIHWVR- | KLLIYSASFLYSGVP |
| | CDR-H3 | QAPGKGLEWVARI- | SRFSGS |
| | Expressed in | YPT- | RSGTDFT- |
| | HL orientation | NGYTRYADSVKGRFT | LTISSLQPEDFA- |
| | | ISADTSKNTAYLQMN | TYYCQQHYT- |
| | | SLRAEDTAVYYCSRW | TPPTFGQGTKVEIKR |
| | | GGDGFYAMDYWGQGT | T (SEQ ID |
| | | LVTVSS (SEQ ID NO: 29) | NO: 30) |
| LH_SSC | Interdomain | EVQLVESGG- | DIQMTQSPSSLSASV |
| | disulfide | GLVQPGGSLRLS- | GDRVTITCRASQDVN |
| | bridge between | CAASGFNIK- | TAVCWYQQKPGKAP- |
| | the CDR-H3 and | DTYIHWVR- | KLLIYSASFLYSGVP |
| | CDR-L1 in position | QAPGKGLEWVARI- | SRFSGSRSGTDFT- |
| | H100B-L34* | YPT- | LTISSLQPEDFA- |
| | Expressed in | NGYTRYADSVKGRFT | TYYCQQHYT- |
| | LH orientation | ISADTSKNTAYLQMN | TPPTFGQGTKVEIKR |
| | | SLRAEDTAVYYCSRW | T (SEQ ID |
| | | GGDGFYCMDYWGQGT | NO: 32) |
| | | LVTVSS (SEQ ID NO: 31) | |
| HL_SSC | Interdomain | EVQLVESGG- | DIQMTQSPSSLSASV |
| | disulfide | GLVQPGGSLRLS- | GDRVTITCRASQDVN |
| | bridge between | CAASGFNIK- | TAVCWYQQKPGKAP- |
| | the CDR-H3 and | DTYIHWVR- | KLLIYSASFLYSGVP |
| | CDR-L1 in position | QAPGKGLEWVARI- | SRFSGSRSGTDFT- |
| | H100B-L34 | YPT- | LTISSLQPEDFA- |
| | Expressed in | NGYTRYADSVKGRFT | TYYCQQHYT- |
| | HL orientation | ISADTSKNTAYLQMN | TPPTFGQGTKVEIKR |
| | | SLRAEDTAVYYCSRW | T (SEQ ID |
| | | GGDGFYCMDYWGQGT | NO: 32) |
| | | LVTVSS (SEQ ID NO: 31) | |
| HL_S-C | Interdomain | EVQLVESGG- | DIQMTQSPSSLSASV |
| | disulfide | GLVQPGGSLRLS- | GDRVTITVRASQDVN |
| | bridge between | CAASGFNIK- | TAVCWYQQKPGKAP- |
| | the CDR-H3 and | DTYIHWVR- | KLLIYSASFLYSGVP |
| | CDR-L1 in positions | QAPGKGLEWVARI- | SRFSGSRSGTDFT- |
| | H100B-L34 * | YPT- | LTISSLQPEDFATY- |
| | Expressed in | NGYTRYADSVKGRFT | YAQQHYT- |
| | HL orientation | ISADTSKNTAYLQMN | TPPTFGQGTKVEIKR |
| | Intradomain disulfide | SLRAEDTAVYYCSRW | T (SEQ ID |
| | bridge removed | GGDGFYCMDYWGQGT | NO: 33) |
| | from V_{L} | LVTVSS (SEQ ID NO: 31) | |
| LH S-C | Interdomain | EVQLVESGG- | DIQMTQSPSSLSASV |
| | disulfide | GLVQPGGSLRLSVAA | GDRVTITCRASQDVN |
| | bridge between | SGFNIKDTYIHWVR- | TAVCWYQQKPGKAP- |
| | the CDR-H3 and | QAPGKGLEWVARI- | KLLIYSASFLYSGVP |
| | CDR-L1 in po- | YPT- | SRFSGSRSGTDFT- |
| | sition H100B- | NGYTRYADSVKGRFT | LTISSLQPEDFA- |
| | L34* | ISADTSKNTAYLQMN | TYYCQQHYT- |
| | Expressed in | SLRAEDTAVY- | TPPTFGQGTKVEIKR |
| | LH orientation | YASRWGG- | T (SEQ ID |
| | Intradomain disulfide | DGFYCMDYWGQGTLV | NO: 32) |
| | bridge removed | TVSS (SEQ ID NO: 34) | |
| | from V_{H} | N | |
| LH_SS+ | Interdomain | EVQLVESGG- | DIQMTQSPSSLSASV |
| | disulfide | GLVQPGGSLRLS- | GDRVTITCRASQDVN |
| | bridge in po- | CAASGFNIK- | TAVAWYQQKPGKAP- |
| | sition H44- | DTYIHWVR- | KLLIYSASFLYSGVP |
| | L100* (Weatherhill | QAPGKCLEWVARI- | SRFSGSRSGTDFT- |
| | et al. 2012) | YPT- | LTISSLQPEDFA- |
| | Expressed in | NGYTRYADSVKGRFT | TYYCQQHYT- |
| | LH orientation | ISADTSKNTAYLQMN | TPPTFGCGTKVEIKR |
| | | SLRAEDTAVYYCSRW | T (SEQ ID |
| | | GGDGFYAMDYWGQGT | NO: 36) |
| | | LVTVSS (SEQ ID NO: 35) | |
| HL_SS+ | Interdomain | EVQLVESGG- | DIQMTQSPSSLSASV |
| | disulfide | GLVQPGGSLRLS- | GDRVTITCRASQDVN |
| | bridge in po- | CAASGFNIK- | TAVAWYQQKPGKAP- |
| | sition H44- | DTYIHWVR- | KLLIYSASFLYSGVP |
| | L100* (Weatherhill | QAPGKCLEWVARI- | SRFSGSRSGTDFT- |
| | et al. 2012) | YPT- | LTISSLQPEDFA- |
| | Expressed in | NGYTRYADSVKGRFT | TYYCQQHYT- |
| | HL orientation | ISADTSKNTAYLQMN | TPPTFGCGTKVEIKR |
| | | SLRAEDTAVYYCSRW | T (SEQ ID |
| | | GGDGFYAMDYWGQGT | NO: 36) |
| | | LVTVSS (SEQ ID NO: 35) | |

*Numbering according to Kabat numbering scheme (Prof Andrew C R Martin′s Group at
UCL 2022). The CDR-H3 and CDR-L1 loops are bolded. The replacement of amino acid
residues (Val, Ala, Gly, or Gln) by Cys is marked with italics and underlined.
The replacement of cysteine residues by Val and Ala is double underlined.

### Example 2. Expression of Anti-HER2 ds-scFvs in E. coli

ScFv fragments LH_SS−, LH_SSC, HL_SSC, HL_S-C, and LH_S-C were expressed in the periplasmic space of E. coli in XL1-Blue strain using vector pAK400. ScFv was produced in shake flask cultures in 300 ml of SB medium containing 0.5% glucose, 10 μg/ml tetracycline, and 25 μg/ml chloramphenicol. Cultures were grown at 37° C., 300 rpm and induced at OD600 of 0.5-1.0 with 200 μM IPTG after which scFv was produced o/n at 26° C., 250 rpm. Cells were harvested by centrifugation (15 min 7000 g, 4° C.). To release periplasmic protein from the cells the pellet was resuspended in 30 ml of 20 mM Phosphate buffer pH 7.4, 300 mM NaCl, 0.4 mg/ml lysozyme, 10 mM MgCl₂, 25 U/ml nuclease. After 30 min incubation at room temperature the sample was freeze-thawn three time. Cell lysate was clarified by centrifugation (20 min 2000 g) and supernatant was filtrated using a ø 0.22 μm filter. ScFv was purified from the cell lysate by Ni-NTA affinity chromatography using 0.5 ml Ni-NTA HisPur resin (Thermo Scientific) and followed by preparative size exclusion chromatography (SEC) using Superdex® 75 10/300 GL column (Cytiva, USA) using PBS pH 7.4 as elution buffer. The concentrations of the scFvs in the SEC fractions were quantified by OctetRED384 (ForteBio, USA) utilizing bio-layer interferometry (BLI) technology. In the measurements, the streptavidin biosensors were coated with biotinylated protein L, which binds to scFv. Purity was analyzed by SDS-PAGE.

After the Ni-NTA purification, the products contained a lot of impurities and therefore they were subjected to size exclusion chromatography (SEC) purification. After SEC purification, the yields of the scFvs with an interloop disulfide bridge were over 10-fold lower than that of the wild type (LH_SS−). Yield of scFv after Ni-NTA and SEC purification are shown in Table 2.

TABLE 2
Yields of the scFvs after protein purification in E. coli.

| | Expression by |
|---|---|
| scFv version | E. coli (μg/ml)^{a} |
| LH_SS− (hu4D5 scFV) | 1124 |
| LH_SSC | 86 |
| HL_SSC | 60 |
| HL_S-C | 82 |
| LH_S-C | 83 |

^{a}The expression yields were determined by Octet utilizing bio-layer interferometry (BLI) technology. The streptavidin biosensors were coated with biotinylated protein L which binds scFv. The value represents the value from one independent measurement.

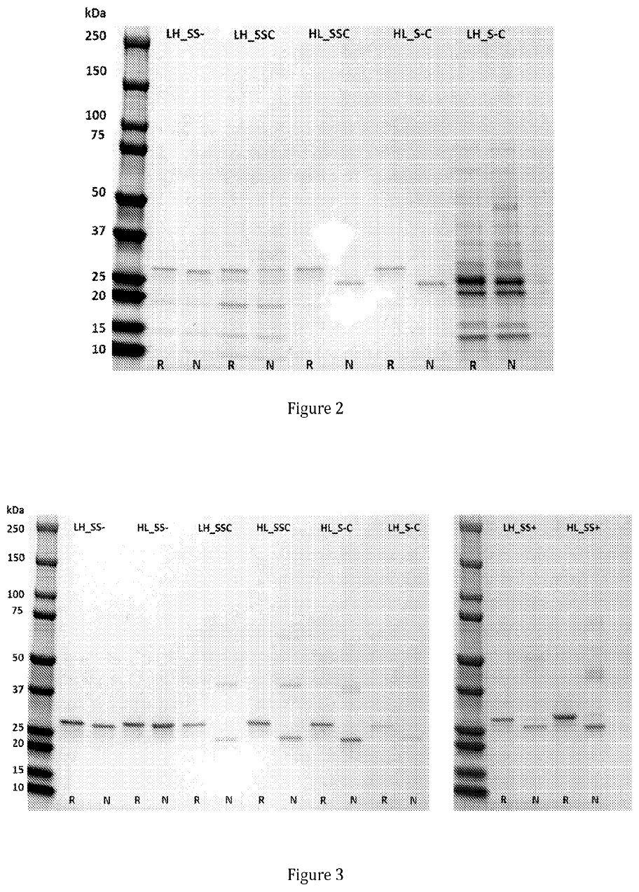

The formation of Interdomain disulfide bridge in scFvs (LH_SS−, LH_SSC, HL_SSC, HL_S-C, and LH_S-C) expressed in E. coli and purified by Ni-NTA and SEC was analysed by SDS-PAGE using both reducing and nonreducing sample buffer. These results are shown in FIG. 2.

Even after SEC purification, the purity levels of scFv versions LH_SSC and LH_S-C remained low. Therefore, the possible interloop disulfide formation could not be assessed. The SEC fractions of LH_SS− also contained some minor impurities. HL_SSC and HL_S-C were able to be purified by Ni-NTA and SEC. The nonreducing SDS-PAGE analysis suggests the formation of an interloop disulfide bridge as the proteins migrate faster (~23 kDa) than their reduced analogs (~28 kDa) (FIG. 2).

### Example 3. Expression of Anti-HER2 ds-scFvs in Mammalian Cells

ScFv fragments LH_SS− (hu4D5 scFv), HL_SS-(hu4D5 scFv), LH_SSC, HL_SSC, HL_S-C, LH_S-C, LH_SS+ and HL_SS+ were expressed in ExpiCHO™ cells (Thermo Scientific) from Vector pTwistCMV Betglobin WPRE Neo (Twist Bioscience).

ExpiCHO cells were transiently transfected with 3 μg of the plasmid using the ExpiFectamine™ CHO (Thermo Scientific) transfection reagent and scFv was produced according to the manufacturer's max titer protocol.

ExpiCHO-S™ cells were cultured with ExpiCHO™ Expression Medium (Thermo Scientific) in 2 ml culturing volume in a six-well plate (Nunclon Delta Surface, Thermo Scientific) covered with breathable sealing tape (Nunc™ Sealing Tapes, Thermo Scientific) on an orbital shaker 125 rpm in a +37° C. incubator with 80% relative humidity and 8% CO₂.

On the day after transfection, 12 μl ExpiFectamine™ CHO enhancer and 320 μl ExpiCHO™ Feed were added to the cells per well. The plates were transferred to a +32° C. incubator with a humidified atmosphere of 5% CO₂ in the air with orbital shaking 125 rpm. On day 5 post-transfection, the second volume of ExpiCHO™ Feed was added to the wells (320 μl/well). The plates were immediately transferred to a +32° C. incubator with a humidified atmosphere of 5% CO₂ in the air with orbital shaking. Culture supernatants were harvested on day 13 after transfection by centrifugation at 5000×g for 30 minutes, then filtrated the supernatant using a ø 0.22 μm filter. ScFv was purified from the cell lysate by Ni-NTA affinity chromatography by using HisPur™ Ni-NTA Spin Columns with a 0.2 ml resin bed (Thermo Scientific). The elution fractions containing most of the protein were combined and buffer was changed to PBS pH7.4 with 10K MWCO Slide-A-Lyzer™ G2 dialysis cassettes (Themro Scientific). Yields of the scFvs after protein purification from ExpiCHO cells were measured by A280 and are shown in Table 3.

All the scFv variants were able to be expressed in ExpiCHO-S™ cells (Table 3). These included the scFv fragment of trastuzumab expressed in both orientations (LH_SS− and HL_SS−), the trastuzumab variants with the interloop disulfide bridge in the position H100B-L34 (LH_SSC and HL_SSC), the trastuzumab variants with the interloop disulfide bridge in the position H100B-L34 and without intradomain disulfide bridge (LH_S-C and HL_S-C), and the trastuzumab variants with disulfide bridge in the position H44-L100 (LH_SS+ and HL_SS+). Based on the literature, H44-L100 has been suggested to serve as a universal location for scFv stabilization (Weatherhill et a. 2012).

The yields of the scFvs expressed in the HL orientation were 2-20-fold higher than those of the scFvs expressed in LH orientation (Table 3).

TABLE 3
Yields of the scFvs after protein purification from ExpiCHO cells.

| | Expression by |
|---|---|
| scFv version | ExpiCHO (μg/ml)^{a} |
| LH_SS− (hu4D5 scFV) | 73 |
| HL_SS− (hu4D5 scFv) | 154 |
| LH_SSC | 27 |
| HL_SSC | 240 |
| HL_S-C | 200 |
| LH_S-C | 10 |
| LH_SS+ | 41 |
| HL_SS+ | 186 |

^{a}The expression yields were determined with absorbance method: A_{280 nm} = 1 correlating to scFv concentration of 0.58 mg/ml. The molar extinction coefficient for LH_SS− was determined by Vector NTI and used to analyze all the scFv constructs. The value represents the value from one independent measurement.

The formation of the interdomain disulfide bridge was evaluated by SDS-PAGE analysis by running the samples of purified scFv versions in reducing and nonreducing sample buffer. Gels were stained with Ready Blue Protein Gel Stain. These results are shown in FIG. 3. Proteins with interdomain disulfide bridges migrate faster in the SDS-PAGE gel in their nonreduced form due to their denser packaging than their analogous variants lacking interdomain disulfide bridge.

Mammalian expressed Ni-NTA purified scFvs did not contain any other proteins as impurities based on the reducing SDS-PAGE analysis (FIG. 3, wells indicated with “R”). However, in nonreduced samples LH_SSC, HL_SSC, HL_S-C, LH_SS+, and HL_SS+ bands with higher molecular weight (~40 and ~60 kDa) were observed, indicating possible disulfide bridge formation between two or more scFvs to form dimers and oligomers, respectively.

The position H100B-L34 enabled complete interloop disulfide bridge formation as evidenced by the nonreducing SDS-PAGE analysis of LH_SSC, HL_SSC, HL_S-C, and LH_S-C: the scFvs migrated faster in the gel (~23 kDa), and the samples did not contain any form where disulfide bridges were absent. However, the position H44-L100 enabled the interdomain disulfide bridge formation only partly, as forms without interdomain disulfide bridge could be detected when samples were run in nonreducing conditions (~28 kDa) (FIG. 3).

### Example 4. Antigen-Binding Properties of Anti-HER2 ds-scFvs

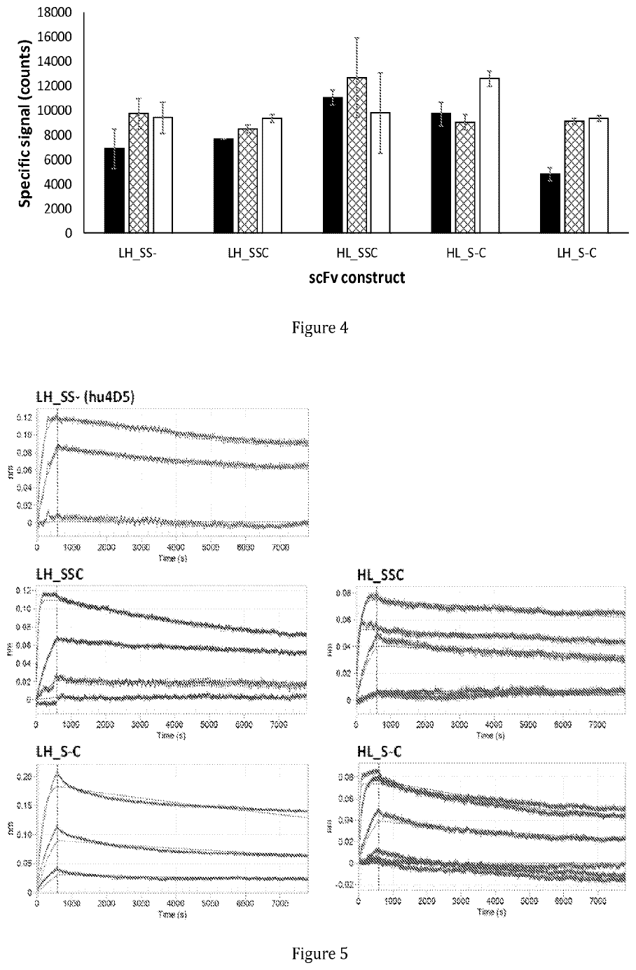

Antigen-binding properties of purified anti-HER2 ds-scFvs were analysed by time-resolved immunofluorometric assay. In the assay biotinylated HER2 was bound to streptavidin coated microtiter wells for 30 min and the plate was washed four times. Then purified scFv was added and incubated for 1 h. After four washes, bound scFv was detected by anti-His tag antibody pentahis (QIAGEN) labelled with Eu-Nl chelate (PerkinElmer, Finland). The plate was washed four times, DELFIA enhancement solution was added and incubated 10 min after which Time-resolved fluorescence was read with Victor multilabel counter (PerkinElmer, Finland). These results are shown in FIG. 4.

As evident from FIG. 4, the binding of the scFv variants with interloop disulfide bridge (LH_SSC, HL_SSC, HL_S-C, and LH_S-C) to HER2 was generally observed to be similar to that of the wild type (LH_S-C) with the concentrations used in the assay. A modest 1.5-fold increase in binding of HL_SSC and HL_S-C at 10 nM concentrations compared to that of the wild type (LH_SS) could be observed. LH_SSC produced a similar signal to the wild type, and LH_S-C showed a 1.5-fold decrease in signal compared to that of the wild type.

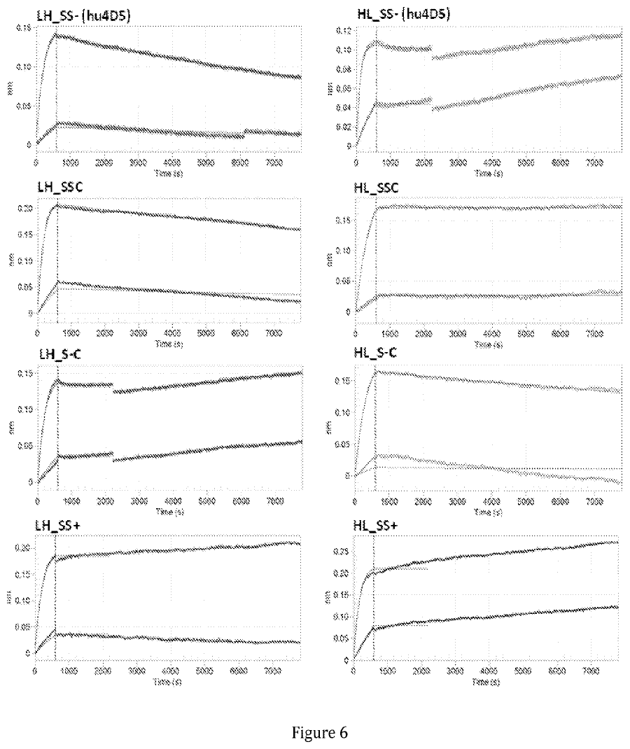

Binding kinetics and preliminary Kd data of purified scFv was obtained with the Octet RED384 (ForteBio) system. Streptavidin coated Biosensors (ForteBio) were loaded with 200 ng/ml of biotinylated HER2 (His, Avitag, Acro Biosystems, USA) for 600 s. ScFv association was followed for 600 s and dissociation for 7200 s. Measurement were done in 100 μl volume in 384 well Tilted bottom plates (ForteBio) at 30 C with shaker speed 1000 rpm and sensor off-set 4 or 6 mm. All measurements were done in PBS pH7.4, 0.1% BSA, 0.05% Tween20. K_{D}, kₐ and k_{d} were calculated using Octet Data analysis software 8.2. These results are shown in FIGS. 5 and 6 and in Tables 4 and 5.

The introduction of the interloop disulfide bridge in H100B-L34 maintained the high binding affinities of the wild type to HER2 (FIGS. 5 and 6, Tables 4 and 5). The trastuzumab scFvs with interdomain disulfide bridge in H44-L100 (LH_SS+ and HL_SS+) also showed to maintain the high binding affinities (K_{d} values at the sub-nanomolar level), which is in line with the previous results obtained for various scFvs stabilized with interdomain disulfide bridge in H44-L100 (Weatherhill et al. 2012; Benschop et al. 2019).

TABLE 4
K_{D}, kₐ, and k_{d} values determined by Octet for the
scFvs expressed by ExpiCHO-S ™ cells

| scFv version | K_{D} (nM)^{a} | kₐ (×10⁴ M⁻¹s⁻¹)^{a} | k_{d} (×10⁻⁵ s⁻¹)^{a} |
|---|---|---|---|
| LH_SS− | 1.380 ± 0.006 | 5.08 ± 0.02 | 6.980 ± 0.012 |
| (hu4D5) | | | |
| HL_SS− | 0.309 ± 0.010 | 9.24 ± 0.05 | 2.85 ± 0.09^{b} |
| (hu4D5) | | | |
| LH_SSC | 0.661 ± 0.005 | 5.60 ± 0.03 | 3.71 ± 0.02 |
| HL_SSC | <0.001 ± 0.004 | 2.030 ± 0.012 | <0.01 |
| HL_S-C | 1.07 ± 0.02 | 2.93 ± 0.05 | 3.12 ± 0.03 |
| LH_S-C | 0.28 ± 0.02 | 5.02 ± 0.03 | 1.41 ± 0.10^{b} |
| LH_SS+ | <0.001 ± 0.03 | 4.83 ± 0.03 | <0.01^{b} |
| HL_SS+ | <0.001 ± 0.03 | 5.84 ± 0.05 | <0.01^{b} |

^{a}K_{D}, kₐ, and k_{d} values determined by monovalent analysis by two scFv concentrations, 10 and 100 nM. The value represents the value from one independent experiment.
^{b}The window for k_{d} value calculations was 0-1600 s.

TABLE 5
K_{D}, kₐ, and k_{d} values determined by Octet
for the scFvs expressed by E. coli

| scFv version | K_{D} (nM) | kₐ (×10⁴ M⁻¹s⁻¹) | k_{d} (×10⁻⁵ s⁻¹) |
|---|---|---|---|
| LH_SS− | 1.230 ± 0.007a | 3.56 ± 0.02a | 4.370 ± 0.013a |
| (hu4D5) | | | |
| LH_SSC | 1.300 ± 0.006^{b} | 5.36 ± 0.02^{b} | 6.95 ± 0.03^{b} |
| HL_SSC | 0.1450 ± 0.0011^{c} | 19.00 ± 0.11^{c} | 2.750 ± 0.012^{c} |
| HL_S-C | 0.437 ± 0.005^{d} | 18.5 ± 0.2^{d} | 8.09 ± 0.03^{d} |
| LH_S-C | 1.500 ± 0.012^{e} | 3.22 ± 0.02^{e} | 4.81 ± 0.02^{e} |

K_{D}, kₐ, and k_{d} values are determined by
athree scFv concentrations 10, 60, and 200 nM by monovalent analysis
^{b}four scFv concentrations 0.2, 2, 10, and 200 nM by bivalent analysis,
^{c}five scFv concentrations 0.2, 0.6, 10, 60, and 200 nM by monovalent analysis,
^{d}five scFv concentrations 0.2, 2, 10, 60, and 200 nM by monovalent, and
^{e}three scFv concentrations 10, 60, and 200 nM by monovalent analysis. The values in the table represent the values from single independent experiments.

### Example 5. Thermostability of Anti-HER2 ds-scFvs

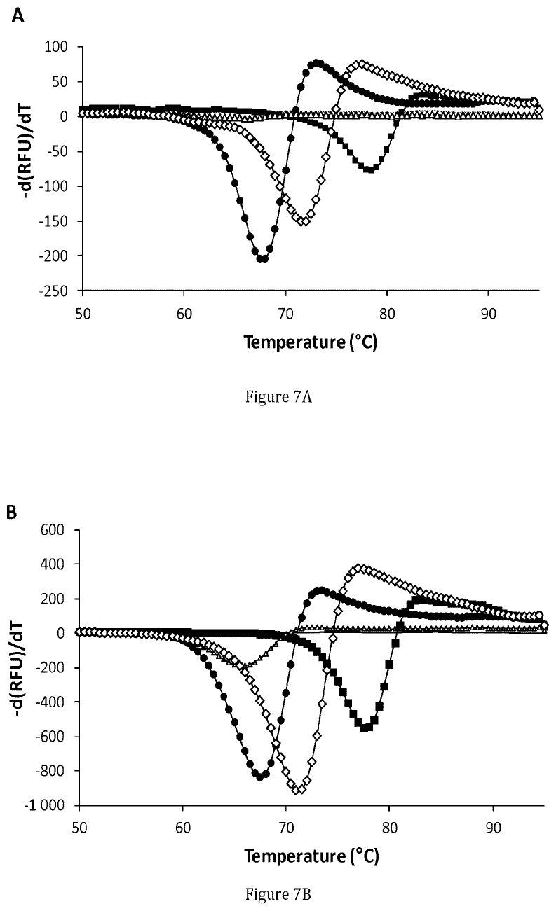

Thermostability was measured with thermofluor assay in a CFX96 Real-time system with C1000 Thermal Cycler and Bio-Rad CFX Manager 3.1 software. The total reaction volume was 25 μl. For the assay 22.5 μl of 5 μM scFv (or 0.35-2 μM for the low yield proteins) in PBS was mixed with 2.5 μl of 50×SYPRO Orange dye (Sigma-Aldrich) diluted in PBS prior to the assay from the 5000× stock. The samples were heated with a PCR system from +25 to +95° C. in +0.5° C. increments and the fluorescence in fluorescence resonance energy transfer (FRET) mode was measured. The mid-point temperature for thermal denaturation (Tₘ) was identified by melt peak analysis plotting the first derivative of the fluorescence emission as a function of temperature (−d(RFU)/dT). These results are shown in FIG. 7 and Tₘ's in Table 6.

Based on the results in Table 6, the scFv variants with interloop disulfide bridge in the position H100B-L34 (LH_SSC and HL_SSC) were significantly more stable than the wild type without interdomain disulfide bridge (LH_SS− and HL_SS−). The Tₘ values +78.5° C. and +77.5° C. for LH_SSC and HL_SSC, respectively were +11 and +9.7° C. higher than the corresponding original scFvs. The removal of the intradomain disulfide bridge from V_{L} (HL_S-C) resulted in a slight decrease in thermal stability (Tₘ=65.7° C.) compared to that of the wild type (Tₘ=67.8° C.).

The interdomain disulfide bridge in position H44-L100 (LH_SS+ and HL_SS+) had a less significant effect on the thermostability than the interloop disulfide bridge in H100B-L34. The Tₘ values were only 3-4° C. higher than those of the wild type but 7° C. lower than those of the variants with an interloop disulfide bridge in the novel H100B-L34 position.

TABLE 6
Thermal stability of the single-chain
variable fragment variable constructs

| | Tₘ (°C) | Tₘ (°C) |
|---|---|---|
| | Expression | Expression |
| scFv version | by Ex-piCHO | by E. coli |
| LH_SS− (hu4D5 scFv) | 67.5 ± 0.0 | 68.0 ± 0.0 |
| | | 42.5 ± 0.0 |
| HL_SS− (hu4D5 scFv) | 67.8 ± 0.2 | nd |
| LH_SSC | 78.5 ± 0.0 | nd |
| HL_SSC | 77.5 ± 0.0 | 75.5 ± 0.0 |
| HL_S-C | 65.7 ± 0.2 | 66.0 ± 0.0 |
| LH_S-C | nd | nd |
| LH_SS+ | 71.7 ± 0.2 | nd |
| HL_SS+ | 71.0 ± 0.0 | nd |

The mid-point temperature for thermal denaturation (Tₘ) of the scFv is determined by thermofluor. The values represent the mean±standard deviation of three replicate measurements.

### Example 6. Phage Display of Anti-HER Ds-scFvs

Phage display of the ds-scFv variants was studied in phagemid vectors pEB32x and pEB3V3. Phage production was done in E. coli XL1-Blue cells. Cells containing the phagemid were inoculated in 20 ml of SB containing 0.5% glucose, 10 μg/ml tetracycline, and 25 μg/ml chloramphenicol and incubated at +37° C., 300 rpm. When OD600 reached 0.4, VCS M13 helper phage was added to 20× multiplicity of infection and cultures were incubated 30 min without shaking. Cells were collected by centrifugation (10 min, 3200 g, 4° C.) and resuspended in 20 ml of SB containing 10 μg/ml tetracycline, and 25 μg/ml chloramphenicol but no glucose. After 1 h shaking at 30 C, 30 μg/ml kanamycin and 100 μM IPTG was added and phage were produced o/n at 26 C, 300 rpm. Cells were removed by centrifugation and phage were precipitated from the supernantant by adding 1/5 volumes of 20% PEG8000, 2.5 M NaCl. Phage was pelleted by centrifugation (20 min, 10000 g, 4 C). The pellet was resuspended in TBS, and after centrifugation to remove any remaining precipitate, phage precipitation with PEG/NaCl was repeated.

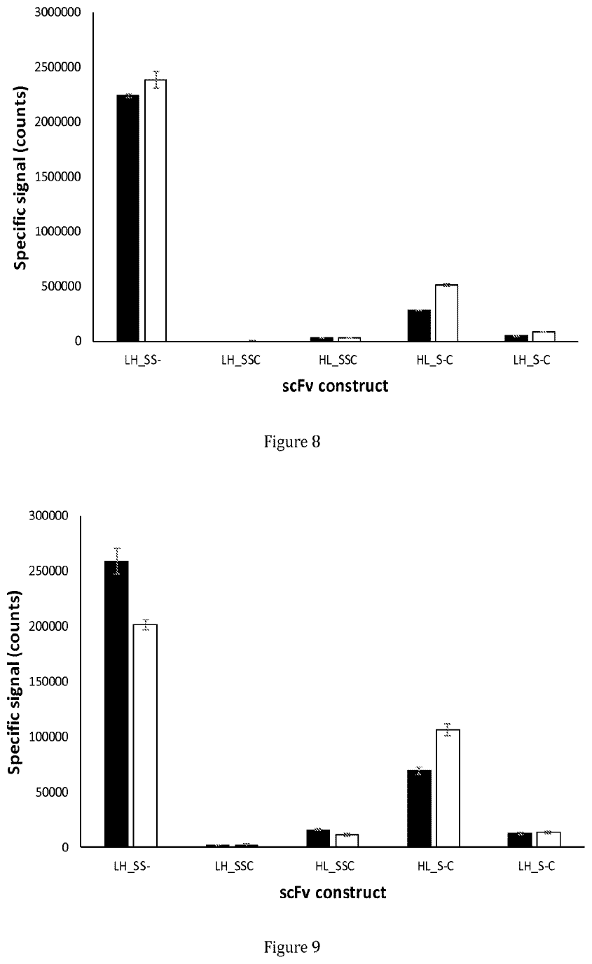

The phage display of scFv was analyzed by immunoassay in which phage binding to biotinylated protein L (FIG. 8) (ProSpec, biotinylated with Ez-link NHS-PEG4 biotin, Thermo) and biotinylated HER2 (FIG. 9) was measured. Protein L is known to bind the Kappa 1 V gene family framework, used in the ds-scFv, in a conformation specific manner. Phage binding to biotinylated HER2 was also measured. All reagents were diluted in Kaivogen red assay buffer. The biotinylated protein L (50 ng/well) and HER2 (10 ng/well) was added onto streptavidin coated microtiter plate wells and incubated for 30 min with slow shaking at room temperature and washed four times. 1e9 phages was added to wells as triplicates and phage binding to streptavidin well was measured as a negative control. Phages were bound for 1 h, RT, with shaking. Then the plate was washed and Eu—Ni-labelled anti-phage antibody (125 ng/ml) was added and incubated for 1 h to detect the bound phages. The plate was again washed 4 times, DELFIA enhancement solution was added and incubated for 10 min after which time-resolved fluorescence was read by Victor 1420 Multilabel Counter (PerkinElmer, Finland). For the assay, phage titer was measured by immunoassay based on phage binding to uncoated maxisorb plate. Bound phages were detected with Eu-labelled anti-phage antibody as described above. These results are presented in FIGS. 8 and 9.

Highest phage display measured from phage binding to protein L was observed with the wild type scFv (LH_SS−) containing only the native intradomain disulfide bridges (FIG. 8). Addition of the artificial interdomain disulfide bridge between CDR-L1 and —H3 in LH_SSC phages having both native intradomain disulfide bridges led to no display of scFv on phage surface. However, phage display was established when the intradomain disulfide bridge in V_{L} was removed from ds-scFv having V_{H}-V_{L} domain order (the HL_S-C variant). Phages that displayed folded scFv also recognized HER2. The signal from binding to protein L and HER2 was 27% and 53% of those of the corresponding wild type phages, respectively (FIGS. 8 and 9).

### Example 7. Construction and Validation of a CDR-H3 Library

The possibility to use the disulfide stabilized scFv as a framework in antibody library was investigated. The ds-scFv variant HL_S-C which was shown to be functionally displayed on phages (FIGS. 8 and 9) was used as a framework in a library where the WGGDGFY (SEQ ID NO:47) sequence of the CDR-H3 loop was diversified with the NNS codon. In addition, various CDR-H3 loop lengths (13, 14, 15, 17, and 19 amino acids) were introduced in the library with similar randomization. The libraries of different loop lengths were constructed and studied separately.

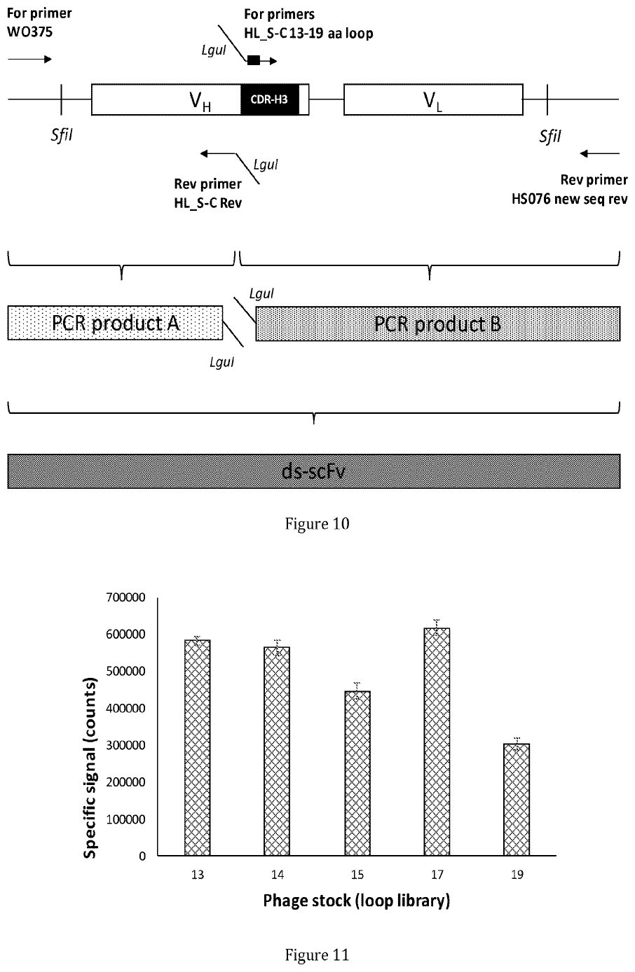

Libraries were constructed by oligonucleotide directed PCR mutagenesis. ScFv was amplified in two PCR fragments that added an LguI site for joining the fragments in FASTR type cloning. NNS codons were added to one of the fragments by randomized oligos FIG. 10. The two fragments were then PCR purified, digested with LguI and joined by T4 DNA ligase to full scFv, after which the product was further amplified, PCR purified, digested with SfiI and cloned in to vector pEB32x. Primers used in library construction are presented in Table 7 and library construction strategy in FIG. 10. In transformation of the ligated library in E. coli XL1-Blue cells, a library size with 5.7×10⁶-4.4×10⁷ transformants was obtained.

TABLE 7
Primers used in the PCR reactions

| | For/ | |
|---|---|---|
| Primer | Rev | Sequence (5′ to 3′)^{a} |
| HL_S-C 13 | For | ACCATT- |
| aa loop | | GCTAGCTCTTCCCGTNNSNNSNNSNNSNNS |
| | | NNSNNSTGTATGGACTACTGGGGTCAG |
| | | (SEQ ID NO: 37) |
| HL S-C 14 | For | ACCATT- |
| aa loop | | GCTAGCTCTTCCCGTNNSNNSNNSNNSNNS |
| | | NNSNNSNNSTGTATGGACTACTGGGGTCAG |
| | | (SEQ ID NO: 38) |
| HL_S-C 15 | For | ACCATT- |
| aa loop | | GCTAGCTCTTCCCGTNNSNNSNNSNNSNNS |
| | | NNSNNSNNSNNSTGTATGGAC- |
| | | TACTGGGGTCAG |
| | | (SEQ ID NO: 39) |
| HL_S-C 17 | For | ACCATT- |
| aa loop | | GCTAGCTCTTCCCGTNNSNNSNNSNNSNNS |
| | | NNSNNSNNSNNSNNSNNSTGTATGGAC- |
| | | TACTGGGGTCAG |
| | | (SEQ ID NO: 40) |
| HL_S-C 19 | For | ACCATT- |
| aa loop | | GCTAGCTCTTCCCGTNNSNNSNNSNNSNNS |
| | | NNSNNSNNSNNSNNSNNSNNSNNSTGTATG |
| | | GACTACTGGGGTCAG |
| | | (SEQ ID NO: 41) |
| WO375 | For | TCACACAGGAAACAGCTATGAC |
| | | (SEQ ID NO: 42) |
| HL_S-C Rev | Rev | ACCATTGCTAGCTCTTCAACGGGAACAG- |
| | | TAGTAGACGGCAG |
| | | (SEQ ID NO: 43) |
| HS076 new | Rev | ACCTGCAATCTGAACGTGACGAC |
| seq rev | | CGAGATAGGGTTGAGTG |
| | | (SEQ ID NO: 44) |

^{a}Hybridizing region underlined. LguI recognition site
is in bold.

To address the possibility of exploiting the novel phage antibody library with interloop disulfide bond as a source of discovery of novel binders against HER2, the loop libraries were enriched against HER2 through three consecutive rounds of selection on a target antigen by panning. Biotinylated HER was immobilized onto streptavidin-coated M280 dynabeads. After four washes phages were bound for 1 h. The beads were washed four times and bound phages were eluted with trypsin. New phage stocks were produced by infecting E. coli XL1-Blue cells with the eluted phages, and after growing the cells to OD600 of 0.4, with VCS M13 helper phage. After o/n production at 26° C., 250 rpm phage were purified the culture medium by two subsequent PEG/NaCl precipitations.

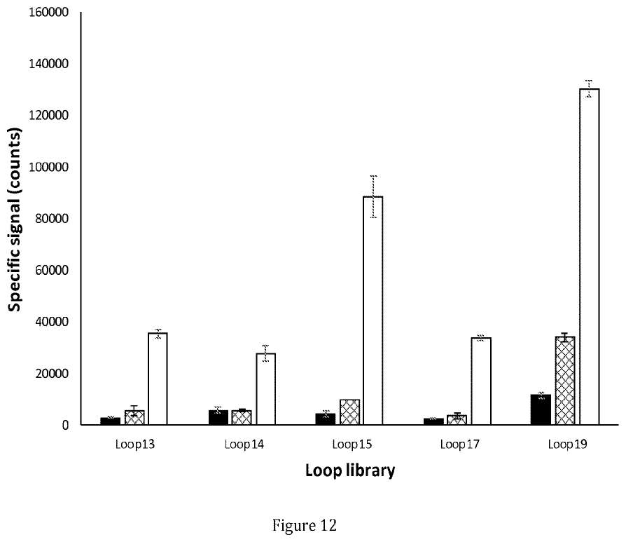

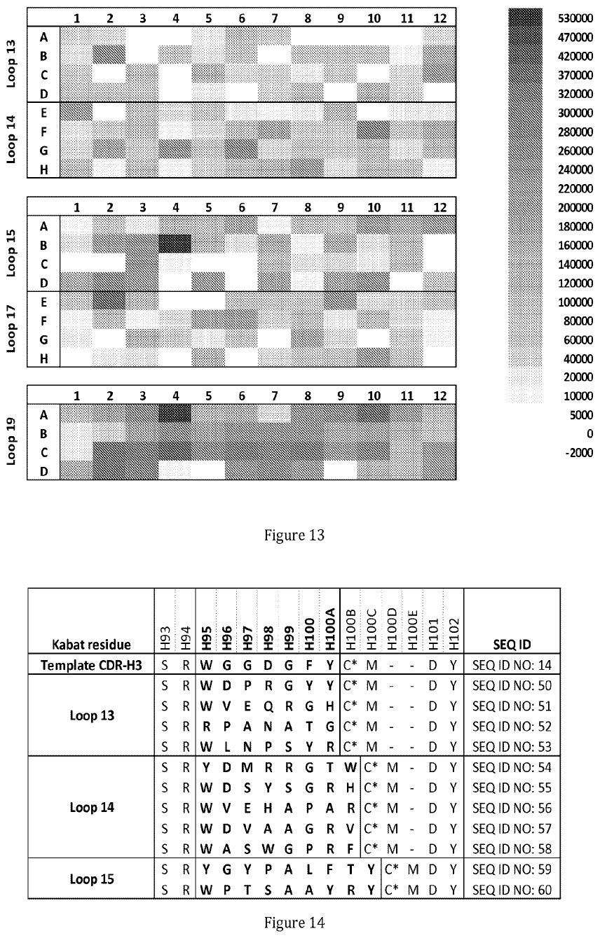

Unselected phage libraries were analysed by immunoassay for binding to protein L. These results are presented in FIG. 11. Phage stocks produced after each panning round were tested by phage immunoassay for binding to HER2. These results are presented in FIG. 12. 48 clones from each library were picked up after three rounds of panning and single phage clones were produced o/n on 96-well cell culture plate. Cells were removed by centrifugation and culture supernatant containing phages was tested in phage immunoassay for binding to HER2. The HER2 bound phages were detected by europium labeled anti-VCS M13 antibody. These results are presented in FIG. 13. From each library a couple of randomly selected clones showing binding to HER2 were sequenced. Some of the sequences are presented in FIG. 14.

Each of the unselected CDR-H3 randomized loop libraries showed binding to protein L (FIG. 11) indicating that also scFv with the alternate loop length was folded correctly and displayed on the phage.

When the library was panned against HER2, the specific signal in phage immunoassay gradually increased through panning rounds in each loop library (FIG. 12), showing the enrichment of (specific) binders against HER2.

After three rounds of panning the number of HER2 positive clones (S/B>5) in the 13, 14, 15, 16, 17, and 19 aa loop libraries was 22/48, 30/48, 28/48, 26/48 and 42/19, respectively (FIG. 13). Sequencing some randomly selected HER2 positive individual clones from 13, 14, and 15 loop libraries (FIG. 14) showed that new sequences were obtained.

It is obvious to a person skilled in the art that with the advancement of technology, the basic idea of the invention may be implemented in various ways. The invention and its embodiments are thus not limited to the examples described above, instead they may vary within the scope of the claims.

### REFERENCES

Weatherill, E. E., Cain, K. L., Heywood, S. P., Compson, J. E., Heads, J. T., Adams, R. & Humphreys, D. P. (2012) Towards a universal disulfide stabilised single chain Fv format: importance of interchain disulphide bond location and VL-VH orientation. Protein Eng Des Sel 25: 321-329
Benschop, R. J., Chow, C. K., Tian, Y., Nelson, J., Barmettler, B., Atwell, S., Clawson, D., Chai, Q., Jones, B., Fitchett, J., Torgerson, S., Ji, Y., Bina, H., Hu, N., Ghanem, M., Manetta, J., Wroblewski, V. J., Lu, J. & Allan, B. W. (2019) Development of tibulizumab, a tetravalent bispecific antibody targeting BAFF and IL17A for the treatment of autoimmune disease. MAbs 11:1175-1190
Krebber A, Bornhauser S, Burmester J, Honegger A, Willuda J, Bosshard H R, Plückthun A. (1997) Reliable cloning of functional antibody variable domains from hybridomas and spleen cell repertoires employing a reengineered phage display system. J. Immunol. Methods. 201(1):35-55.
Huovinen T, Syrjänpää M, Sanmark H, Brockmann E C, Azhayev A, Wang Q, Vehniainen M, Lamminmaki U. (2013) Two ScFv antibody libraries derived from identical VL-VH framework with different binding site designs display distinct binding profiles. Protein Eng Des Sel. 26(10):683-93.
Vazquez-Lombardi R, Nevoltris D, Luthra A, Schofield P, Zimmermann C, Christ D. (2018) Transient expression of human antibodies in mammalian cells. Nat. Protoc. 13(1):99-117.

1.-29. (canceled)

30. A disulfide-stabilized single-chain fragment variable (ds-scFv) comprising:
a heavy chain variable domain (V_{H}) comprising complementary determining regions CDR-H1 and CDR-H2 and CDR-H3, and
a light chain variable domain (V_{L}) comprising complementary determining regions CDR-L1, CDR-L2 and CDR-L3,
wherein the V_{H} and the V_{L} are joined by a peptide linker in either orientation, and
wherein the CDR-L1 and the CDR-H3 are attached to each other through an interdomain disulfide bridge, and wherein either the V_{H} or the V_{L} has been engineered to lack a native intradomain disulfide bridge.

31. The ds-scFv according to claim 30, wherein the interdomain disulfide bridge is artificial.

32. The ds-scFv according to claim 30, wherein the interdomain disulfide bridge is formed between a cysteine residue in the CDR-H3 at position −4 counted from conserved tryptophan H103 of a framework region following the CDR-H3 (FR-H4) and a cysteine residue at position L34 in the CDR-L1, all numberings being according to the Kabat numbering scheme.

33. The ds-scFv according to claim 30, wherein the native intradomain disulfide bridge lacking from the variable domain is located after the peptide linker.

34. The ds-scFv according to claim 30, wherein the native intradomain disulfide bridge lacking from the variable domain is either a bridge formed between cysteine residues L23 and L88 in V_{L} or a bridge between cysteine residues H22 and H92 in V_{H} according to the Kabat numbering scheme.

35. The ds-scFv according to claim 30, wherein the V_{H} domain comprises a framework comprising FR-H1 of SEQ ID NO:1, FR-H2 of SEQ ID NO:2, FR-H3 of SEQ ID NO:3 and FR-H4 of SEQ ID NO:4, and the V_{L} domain comprises a framework comprising FR-L1 of SEQ ID NO:5, FR-L2 of SEQ ID NO:6, FR-L3 of SEQ ID NO:7 and FR-L4 of SEQ ID NO:8 engineered such that either one or both of amino residues 22 of SEQ ID NO:1 and 30 of SEQ ID NO:3 are not cysteines or one or both of amino acid residues 23 of SEQ ID NO:5 and 32 of SEQ ID NO:7 are not cysteines; or wherein the V_{H} domain comprises an amino acid sequence set forth in SEQ ID NO:48 or a functionally equivalent conservative sequence variant thereof, and the V_{L} domain comprises an amino acid sequence set forth in SEQ ID NO:49 or a functionally equivalent sequence variant thereof engineered such that either one or both of the amino acid residues at positions 22 and 98 of SEQ ID NO:48 are not cysteines or such that either one or both of the amino acid residues at positions 23 and 88 of SEQ ID NO:49 are not cysteines.

36. The ds-scFv according to claim 30, further engineered such that the native intradomain disulfide bridge lacking within either the V_{H} or the V_{L} has been reintroduced.

37. The ds-scFv according to claim 36, wherein the V_{H} domain comprises a framework comprising FR-H1 of SEQ ID NO:1, FR-H2 of SEQ ID NO:2, FR-H3 of SEQ ID NO:3 and FR-H4 of SEQ ID NO:4, and the V_{L} domain comprises a framework comprising FR-L1 of SEQ ID NO:5, FR-L2 of SEQ ID NO:6, FR-L3 of SEQ ID NO:7 and FR-L4 of SEQ ID NO:8; or wherein the V_{H} domain comprises an amino acid sequence set forth in SEQ ID NO:48 or a functionally equivalent conservative sequence variant thereof, and the V_{L} domain comprises an amino acid sequence set forth in SEQ ID NO:49 or a functionally equivalent sequence variant thereof.

38. The ds-scFv according to claim 30, wherein the ds-scFv is an anti-HER2 ds-scFv, preferably wherein the CDR-H1 has an amino acid sequence of SEQ ID NO:12, the CDR-H2 has an amino acid sequence of SEQ ID NO:13, the CDR-H3 has an amino acid sequence of SEQ ID NO:14, the CDR-L1 has an amino acid sequence of SEQ ID NO:15, the CDR-L2 has an amino acid sequence of SEQ ID NO:16 and the CDR-L3 has an amino acid sequence of SEQ ID NO:17.

39. The ds-scFv according to claim 38, wherein the anti-HER2 ds-scFv comprises an amino acid sequence of SEQ ID NO: 20 or 21.

40. A molecular entity comprising one or more ds-scFv units according to claim 30.

41. The molecular entity according to claim 40, wherein the molecular entity is a bispecific antibody, a diabody, a multispecific antibody, a CAR-T cell, a bispecific T-cell engager (BiTE), a construct wherein the one or more ds-scFv units is fused to a moiety selected from a fragment crystallizable (Fc) part of an antibody and alternative protein scaffold-based affinity reagents, a pharmaceutically active agent, a drug, a radioisotope, an enzyme or a chelating agent.

42. A particle displaying on its surface the ds-scFv according to claim 30.

43. The particle according to claim 42, wherein the particle is a phage particle, a yeast cell, a bacterial cell, a mammalian cell or a ribosome.

44. A method of making a library of particles, the library displaying a plurality of different ds-scFvs according to claim 30, the method comprising:
i) engineering a plurality of first nucleic acids that encode different heavy chain variable domain (V_{H}) polypeptides all having a cysteine residue at position −4 counted from conserved tryptophan H103 according to the Kabat numbering scheme;
ii) engineering one or a plurality of second nucleic acids that encode different light chain variable domain (V_{L}) polypeptides all having a cysteine residue at position L34 according to the Kabat numbering scheme;
iii) cloning each one of the plurality of first nucleic acids and one of the second nucleic acids into an expression vector, the first and the second nucleic acids being in either order and intervened by a nucleic acid encoding a peptide linker, thereby resulting in a plurality of different vectors; and
iv) expressing the plurality of different vectors of step iii) on particles, thereby generating a first library of particles, each particle displaying a different ds-scFv polypeptide comprising a V_{H} and a V_{L} segment encoded by the plurality of nucleic acids of steps i) and ii), wherein
the plurality of either the first or the second nucleic acids is engineered to lack a native intradomain disulfide bridge in the encoded polypeptide.

45. The method according to claim 44, wherein the native intradomain disulfide bridge is lacking from the variable domain placed after the peptide linker in the encoded polypeptide.

46. The method according to claim 44, wherein, the first library of particles is a phage display library.

47. The method according to claim 44, wherein the method comprises introducing further diversity into a subset of the first library of particles having desired target-binding properties, preferably by mutagenesis or domain shuffling of either the V_{H} or V_{L}, preferably by shuffling of the variable domain located after the peptide linker, thereby creating a second library of particles.

48. The method according to claim 47, wherein the mutagenesis or the domain shuffling results in reintroduction of the native intradomain disulfide bridge engineered to lack either from the first or the second nucleic acid.

49. The method according to claim 44, wherein the particles of the second library of particles are cell particles, preferably yeast or mammalian cell, more preferably mammalian cells.

50. The ds-scFv according to claim 30, wherein the V_{H} precedes the peptide linker and the native intradomain disulfide bridge is lacking from the V_{L}.


SEQUENCE LISTING (60 sequences)

SEQ ID NO: 1 - protein, 30 aa, synthetic construct
  DISULFID 22: note = intradomain (between FR-H1 and FR-H3)
EVQLVESGGGLVQPGGSLRLSCAASGFNIK

SEQ ID NO: 2 - protein, 14 aa, synthetic construct
WVRQAPGKGLEWVA

SEQ ID NO: 3 - protein, 30 aa, synthetic construct
  DISULFID 30: note = intradomain (between FR-H3 and FR-H1)
RFTISADTSKNTAYLQMNSLRAEDTAVYYC

SEQ ID NO: 4 - protein, 11 aa, synthetic construct
WGQGTLVTVSS

SEQ ID NO: 5 - protein, 23 aa, synthetic construct
  DISULFID 23: note = intradomain (between FR-L1 and FR-L3)
DIQMTQSPSSLSASVGDRVTITC

SEQ ID NO: 6 - protein, 15 aa, synthetic construct
WYQQKPGKAPKLLIY

SEQ ID NO: 7 - protein, 32 aa, synthetic construct
  DISULFID 32: note = intradomain (between FR-L3 and FR-L1)
GVPSRFSGSRSGTDFTLTISSLQPEDFATYYC

SEQ ID NO: 8 - protein, 10 aa, synthetic construct
FGQGTKVEIK

SEQ ID NO: 9 - protein, 15 aa, synthetic construct
GGGGSGGGGSGGGGS

SEQ ID NO: 10 - protein, 20 aa, synthetic construct
GGGGSGGGGSGGGGSGGGGS

SEQ ID NO: 11 - protein, 20 aa, synthetic construct
GGGGSGAGGSGGGGTGGGGS

SEQ ID NO: 12 - protein, 5 aa, synthetic construct
DTYIH

SEQ ID NO: 13 - protein, 17 aa, synthetic construct
RIYPTNGYTRYADSVKG

SEQ ID NO: 14 - protein, 13 aa, synthetic construct
  DISULFID 10: note = interdomain (between CDR-H3 and CDR-L1)
SRWGGDGFYCMDY

SEQ ID NO: 15 - protein, 11 aa, synthetic construct
  DISULFID 11: note = interdomain (between CDR-L1 and CDR-H3)
RASQDVNTAVC

SEQ ID NO: 16 - protein, 7 aa, synthetic construct
SASFLYS

SEQ ID NO: 17 - protein, 9 aa, synthetic construct
QQHYTTPPT

SEQ ID NO: 18 - protein, 13 aa, synthetic construct
SRWGGDGFYAMDY

SEQ ID NO: 19 - protein, 11 aa, synthetic construct
RASQDVNTAVA

SEQ ID NO: 20 - protein, 249 aa, synthetic construct
  DISULFID 22..96: note = intradomain (between FR-H1 and FR-H3)
  DISULFID 163..228: note = intradomain (between FR-L1 and FR-L3)
  DISULFID 106..174: note = interdomain (between CDR-H3 and CDR-L1)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYCMDYWGQGTLVTVSSGGGGSGAGGSGGGGTGGGGSDIQMTQSPSSLSASVGDRVTITCRASQDVNTAVCWYQQKPGKAPKLLIYSASFLYSGVPSRFSGSRSGTDFTLTISSLQPEDFATYYCQQHYTTPPTFGQGTKVEIKRT

SEQ ID NO: 21 - protein, 249 aa, synthetic construct
  DISULFID 23..88: note = intradomain (between FR-L1 and FR-L3)
  DISULFID 151..225: note = intradomain (between FR-H1 and FR-H3)
  DISULFID 34..235: note = interdomain (between CDR-L1 and CDR-H3)
DIQMTQSPSSLSASVGDRVTITCRASQDVNTAVCWYQQKPGKAPKLLIYSASFLYSGVPSRFSGSRSGTDFTLTISSLQPEDFATYYCQQHYTTPPTFGQGTKVEIKRTGGGGSGAGGSGGGGTGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYCMDYWGQGTLVTVSS

SEQ ID NO: 22 - protein, 22 aa, synthetic construct
MKSLLPTAAAGLLLLAAQPAMA

SEQ ID NO: 23 - protein, 22 aa, synthetic construct
MKYLLPTVVVGLLLLAAQPAMA

SEQ ID NO: 24 - DNA, 66 bp, synthetic construct
  source 1..66: mol_type = other DNA
atgaagtaccttctaccgacggtagtcgttggattgttattactcgcggcccagccggccatggcg

SEQ ID NO: 25 - protein, 22 aa, synthetic construct
MKYLLPTAAAGLLLLAAQPAMA

SEQ ID NO: 26 - DNA, 66 bp, synthetic construct
  source 1..66: mol_type = other DNA
atgaaatacctattgcctacggcagccgctggattgttattactcgcggcccagccggccatggcg

SEQ ID NO: 27 - skipped

SEQ ID NO: 28 - protein, 20 aa, synthetic construct
MVLQTQVFISLLLWISGAYG

SEQ ID NO: 29 - protein, 120 aa, synthetic construct [sequence omitted]

SEQ ID NO: 30 - protein, 109 aa, synthetic construct
  DISULFID 22..88: note = intradomain (between FR-L1 and FR-L3)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 31 - protein, 120 aa, synthetic construct
  DISULFID 22..96: note = intradomain (between FR-H1 and FR-H2)
  DISULFID 106: note = interdomain (between CDR-H3 and CDR-L1)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYCMDYWGQGTLVTVSS

SEQ ID NO: 32 - protein, 109 aa, synthetic construct
  DISULFID 23..88: note = intradomain (between FR-L1 and FR-L3)
  DISULFID 34: note = interdomain (between CDR-L1 and CDR-H3)
DIQMTQSPSSLSASVGDRVTITCRASQDVNTAVCWYQQKPGKAPKLLIYSASFLYSGVPSRFSGSRSGTDFTLTISSLQPEDFATYYCQQHYTTPPTFGQGTKVEIKRT

SEQ ID NO: 33 - protein, 109 aa, synthetic construct
  DISULFID 34: note = interdomain (between CDR-L1 and CDR-H3)
DIQMTQSPSSLSASVGDRVTITVRASQDVNTAVCWYQQKPGKAPKLLIYSASFLYSGVPSRFSGSRSGTDFTLTISSLQPEDFATYYAQQHYTTPPTFGQGTKVEIKRT

SEQ ID NO: 34 - protein, 120 aa, synthetic construct
  DISULFID 106: note = interdomain (between CDR-H3 and CDR-L1)
EVQLVESGGGLVQPGGSLRLSVAASGFNIKDTYIHWVRQAPGKGLEWVARIYPTNGYTRYADSVKGRFTISADTSKNTAYLQMNSLRAEDTAVYYASRWGGDGFYCMDYWGQGTLVTVSS

SEQ ID NO: 35 - protein, 120 aa, synthetic construct
  DISULFID 22..96: note = intradomain (between FR-H1 and FR-H3)
  DISULFID 44: note = interdomain (between CDR-H3 and CDR-L1)
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 36 - protein, 109 aa, synthetic construct
  DISULFID 23..88: note = intradomain (between FR-L1 and FR-L3)
  DISULFID 100: note = interdomain (between CDR-L1 and CDR-H3)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKRT

SEQ ID NO: 37 - protein, 63 aa, synthetic construct
ACCATTGCTAGCTCTTCCCGTNNSNNSNNSNNSNNSNNSNNSTGTATGGACTACTGGGGTCAG

SEQ ID NO: 38 - protein, 66 aa, synthetic construct
ACCATTGCTAGCTCTTCCCGTNNSNNSNNSNNSNNSNNSNNSNNSTGTATGGACTACTGGGGTCAG

SEQ ID NO: 39 - protein, 69 aa, synthetic construct
ACCATTGCTAGCTCTTCCCGTNNSNNSNNSNNSNNSNNSNNSNNSNNSTGTATGGACTACTGGGGTCAG

SEQ ID NO: 40 - protein, 75 aa, synthetic construct
ACCATTGCTAGCTCTTCCCGTNNSNNSNNSNNSNNSNNSNNSNNSNNSNNSNNSTGTATGGACTACTGGGGTCAG

SEQ ID NO: 41 - protein, 81 aa, synthetic construct
ACCATTGCTAGCTCTTCCCGTNNSNNSNNSNNSNNSNNSNNSNNSNNSNNSNNSNNSNNSTGTATGGACTACTGGGGTCAG

SEQ ID NO: 42 - protein, 22 aa, synthetic construct
TCACACAGGAAACAGCTATGAC

SEQ ID NO: 43 - protein, 41 aa, synthetic construct
ACCATTGCTAGCTCTTCAACGGGAACAGTAGTAGACGGCAG

SEQ ID NO: 44 - protein, 40 aa, synthetic construct
ACCTGCAATCTGAACGTGACGACCGAGATAGGGTTGAGTG

SEQ ID NO: 45 - DNA, 747 bp, synthetic construct
  source 1..747: mol_type = other DNA
gaagtgcagctggtagaaagtgggggtggccttgtacagccaggggggtctctgcgtctctcttgtgcagccagtggttttaacatcaaagatacgtacattcattgggttcgccaggcccctgggaaagggctggaatgggttgctcgcatctaccccactaatggatatacgcgttatgcagattctgtgaaaggccgctttaccatttctgcggatacgtctaaaaacacagcgtacctgcaaatgaacagcctgcgtgccgaagacactgccgtctactactgttcccgttggggtggcgatggtttctactgtatggactactggggtcagggtacactagtcaccgtgagctcgggtggtggcggcagtggcgccggtgggtctggaggtggcggtaccggtggcggagggtcagatattcagatgacccagagtccttcttctctgagtgcaagtgtaggtgatcgtgttaccattacttgccgcgcatcacaggatgttaacaccgccgtatgttggtaccagcagaaacctgggaaagccccaaaactcctgatttactctgcatctttcctttactcaggggttccttcccgcttttcaggctcacgcagtgggacggactttactctcaccattagctcactgcaaccagaagacttcgcaacttattattgtcaacagcactatactaccccaccgacctttggccaggggacgaaagtcgagattaaacgcacg

SEQ ID NO: 46 - DNA, 747 bp, synthetic construct
  source 1..747: mol_type = other DNA
gatattcagatgacccagagtccttcttctctgagtgcaagtgtaggtgatcgtgttaccattacttgccgcgcatcacaggatgttaacaccgccgtatgttggtaccagcagaaacctgggaaagccccaaaactcctgatttactctgcatctttcctttactcaggggttccttcccgcttttcaggctcacgcagtgggacggactttactctcaccattagctcactgcaaccagaagacttcgcaacttattattgtcaacagcactatactaccccaccgacctttggccaggggacgaaagtcgagattaaacgcacaggtggtggcggcagtggcgccggtgggtctggaggtggcggtaccggtggcggagggtcagaagtgcagctggtagaaagtgggggtggccttgtacagccaggggggtctctgcgtctctcttgtgcagccagtggttttaacatcaaagatacgtacattcattgggttcgccaggcccctgggaaagggctggaatgggttgctcgcatctaccccactaatggatatacgcgttatgcagattctgtgaaaggccgctttaccatttctgcggatacgtctaaaaacacagcgtacctgcaaatgaacagcctgcgtgccgaagacactgccgtctactactgttcccgttggggtggcgatggtttctactgtatggactactggggtcagggtacactagtcaccgtgagctcg

SEQ ID NO: 47 - protein, 7 aa, synthetic construct
WGGDGFY

SEQ ID NO: 48 - protein, 130 aa, synthetic construct
  REGION 1..30: note = FR-H1
  REGION 31..35: note = CDR-H1
  REGION 36..49: note = FR-H2
  REGION 50..68: note = CDR-H2
  REGION 69..98: note = FR-H3
  REGION 99..119: note = CDR-H3
  REGION 120..130: note = FR-H4
  VARIANT 31..35: note = X is target-specific undefined amino acid
  VARIANT 50..68: note = X is target-specific undefined amino acid
  VARIANT 50..52: note = any one of these amino acids may be missing
  VARIANT 99..115: note = X is target-specific undefined amino acid
  VARIANT 99..107: note = any one of these amino acids may be missing
  VARIANT 117..119: note = X is target-specific undefined amino acid
  DISULFID 22..98: note = intradomain (between FR-H1 and FR-H3)
  DISULFID 116: note = interdomain (between CDR-H3 and CDR-L1)
EVQLVESGGGLVQPGGSLRLSCAASGFNIKXXXXXWVRQAPGKGLEWVAXXXXXXXXXXXXXXXXXXXRFTISADTSKNTAYLQMNSLRAEDTAVYYCXXXXXXXXXXXXXXXXXCXXXWGQGTLVTVSS

SEQ ID NO: 49 - protein, 109 aa, synthetic construct
  REGION 1..23: note = FR-L1
  REGION 24..34: note = CDR-L1
  REGION 35..49: note = FR-L2
  REGION 50..56: note = CDR-L2
  REGION 57..88: note = FR-L3
  REGION 89..99: note = CDR-L3
  REGION 100..109: note = FR-L4
  VARIANT 24..33: note = X is target-specific undefined amino acid
  VARIANT 50..56: note = X is target-specific undefined amino acid
  VARIANT 89..99: note = X is target-specific undefined amino acid
  VARIANT 89..90: note = any one of these amino acids may be missing
  DISULFID 23..88: note = intradomain (between FR-L1 and FR-L3)
  DISULFID 34: note = interdomain (between CDR-L1 and CDR-H3)
DIQMTQSPSSLSASVGDRVTITCXXXXXXXXXXCWYQQKPGKAPKLLIYXXXXXXXGVPSRFSGSRSGTDFTLTISSLQPEDFATYYCXXXXXXXXXXXFGQGTKVEIK

SEQ ID NO: 50 - protein, 13 aa, synthetic construct
SRWDPRGYYCMDY

SEQ ID NO: 51 - protein, 13 aa, synthetic construct
SRWVEQRGHCMDY

SEQ ID NO: 52 - protein, 13 aa, synthetic construct
SRRPANATGCMDY

SEQ ID NO: 53 - protein, 13 aa, synthetic construct
SRWLNPSYRCMDY

SEQ ID NO: 54 - protein, 14 aa, synthetic construct
SRYDMRRGTWCMDY

SEQ ID NO: 55 - protein, 14 aa, synthetic construct
SRWDSYSGRHCMDY

SEQ ID NO: 56 - protein, 14 aa, synthetic construct
SRWVEHAPARCMDY

SEQ ID NO: 57 - protein, 14 aa, synthetic construct
SRWDVAAGRVCMDY

SEQ ID NO: 58 - protein, 14 aa, synthetic construct
SRWASWGPRFCMDY

SEQ ID NO: 59 - protein, 15 aa, synthetic construct
SRYGYPALFTYCMDY

SEQ ID NO: 60 - protein, 15 aa, synthetic construct
SRWPTSAAYRYCMDY